(19) United States
(12) Patent Application Publication
(10) Pub. No.: US 2015/0073212 A1
(43) Pub. Date: Mar. 12, 2015

(54) Endoscope Apparatus

(71) Applicant: Semiconductor Energy Laboratory Co., Ltd., Kanagawa-ken (JP)

(72) Inventors: Shunpei Yamazaki, Tokyo (JP); Yoshiharu Hirakata, Ebina (JP)

(21) Appl. No.: 14/481,213

(22) Filed: Sep. 9, 2014

(30) Foreign Application Priority Data
Sep. 11, 2013 (JP) 2013-188612

(51) Int. Cl. A61B 1/07; A61B 1/00; A61B 1/05; A61B 1/06

(52) U.S. Cl. CPC A61B 1/07; A61B 1/0676; A61B 1/00096; A61B 1/05; A61B 1/0684
USPC 600/109; 600/179

(57) ABSTRACT

An endoscope apparatus with which the inside of an inspection object can be observed in detail is provided. In addition, the structure of the endoscope apparatus is simplified. The endoscope apparatus includes a tubular insertion portion having an end portion, and the end portion is provided with a light-emitting device and an optical device. The optical device is provided on an end surface in the end portion, and the light-emitting device is provided along a side surface of the end portion.

### BACKGROUND OF THE INVENTION

1. Field of the Invention

One embodiment of the present invention relates to an endoscope apparatus, particularly to an endoscope apparatus provided with a light-emitting device.

Note that one embodiment of the present invention is not limited to the above technical field. One embodiment of the invention disclosed in this specification and the like relates to an object, a method, and a manufacturing method. One embodiment of the present invention relates to a process, a machine, manufacture, and a composition of matter. Specifically, examples of the technical field of one embodiment of the present invention disclosed in this specification include a semiconductor device, a display device, a light-emitting device, a lighting device, a method for driving any of them, and a method for manufacturing any of them.

Note that the light-emitting device in this specification refers to any device including a light-emitting element and includes a light source (including a lighting device).

2. Description of the Related Art

As an implement for observing the inside of an inspection object, an endoscope apparatus has been known. The endoscope apparatus is provided with a long and narrow insertion portion that can be curved. As the observation implement, for example, a fiberscope including an optical fiber and an electronic endoscope apparatus including a charge coupled device (CCD) camera and the like are given.

In the endoscope apparatus, lighting for lightening the inside of the inspection object is provided at the end surface of the insertion portion. As a structure of the lighting, a method in which a light source such as a xenon lamp is provided outside and light is transmitted to the end surface of the insertion portion, a structure in which a light emitting diode (LED) element is provided at the end surface of the insertion portion, or the like is used. For example, in Patent Document 1, a structure in which a light source device supplying light to an endoscope is provided and a light guide that transmits light from the light source device to an end portion is incorporated in an insertion portion is disclosed.

### REFERENCE

### Patent Document

[Patent Document 1] PCT International Publication No. WO2011/145392

### SUMMARY OF THE INVENTION

In the case where the light source such as the xenon lamp is used as the lighting for lightening the inside of the inspection object, the size of the endoscope apparatus becomes large. On the other hand, with the structure including the LED element, the structure of the endoscope apparatus can be simplified, but it is difficult to improve the color rendering property of illumination light, so that it is also difficult to observe the inside of the inspection object in detail. In addition, when such a light source is used, a light-emitting area is extremely small and light with high directivity is emitted; thus, because of a shadow formed by illuminating the inside of the inspection object, a portion that cannot be observed is generated.

An object of one embodiment of the present invention is to provide an endoscope apparatus with which the inside of an inspection object can be observed in detail. Another object is to simplify the structure of an endoscope apparatus. Another object is to provide an endoscope apparatus which is less likely to produce a shadow. Another object is to provide an endoscope apparatus with which a three-dimensional observation is easily performed. Another object is to provide an endoscope apparatus that can control a direction of light emitted from a light source. Another object is to provide a novel endoscope apparatus. Another object is to provide a novel light-emitting device.

Note that the descriptions of these objects do not disturb the existence of other objects. In one embodiment of the present invention, there is no need to achieve all the objects. Other objects will be apparent from and can be derived from the description of the specification, the drawings, the claims, and the like.

One embodiment of the present invention is an endoscope apparatus including a tubular insertion portion including an end portion. The end portion includes a light-emitting device and an optical device. The optical device is placed on an end surface of the end portion, and the light-emitting device is provided along a side surface of the end portion.

The light-emitting device is preferably a surface light-emitting device.

The optical device is preferably an image sensor or an optical fiber.

The light-emitting device is preferably provided to emit light from the side surface of the end portion to the outside thereof. At this time, in particular, the light-emitting device is preferably provided to cover part of the end surface of the end portion.

Alternatively, it is preferable that the light-emitting device be provided to emit light from the side surface of the end portion to the inside of the end portion, and a reflection member reflecting the light be provided inside the end portion to emit the light from the end surface. At this time, in particular, it is preferable that the light-emitting device be a dual-emission light-emitting device, and the light-emitting device be provided to emit light from the light-emitting device from the side surface of the end portion to the inside and the outside of the end portion.

An image obtained through the optical device preferably has a first mode to observe a moving image and a second mode to take a still image. In the first mode, light whose emission luminance is greater than or equal to 100 cd/cm² and less than 10000 cd/cm² is preferably emitted continuously or intermittently, and in the second mode, light whose emission luminance is greater than or equal to 10000 cd/cm² and less than or equal to 500000 cd/cm² is preferably emitted by being synchronized with shutter speed.

The light-emitting device preferably includes a light-emitting element containing a light-emitting organic compound. At this time, in particular, it is preferable that a display portion displaying an image obtained through the optical device be further included, and a pixel in the display portion include a light-emitting element containing a light-emitting organic compound.

According to one embodiment of the present invention, an endoscope apparatus with which the inside of an inspection object can be observed in detail can be provided. In addition, the structure of an endoscope apparatus can be simplified. Moreover, it is possible to provide an endoscope apparatus which is less likely to produce a shadow, an endoscope apparatus with which a three-dimensional observation is easily performed, or an endoscope apparatus that can control a direction of light emitted from a light source. Note that one embodiment of the present invention is not limited to the above effects. For example, depending on circumstances, one embodiment of the present invention might produce another effect or might not produce any of the above effects.

### BRIEF DESCRIPTION OF THE DRAWINGS

In the accompanying drawings:

FIGS. 1A to 1C illustrate a structural example of an endoscope apparatus of one embodiment;

FIG. 2 illustrates a structural example of an endoscope apparatus of one embodiment;

FIGS. 3A and 3B illustrate structural examples of an endoscope apparatus of one embodiment;

FIGS. 4A to 4C illustrate structural examples of an endoscope apparatus of one embodiment;

FIGS. 5A and 5B illustrate structural examples of an endoscope apparatus of one embodiment;

FIGS. 6A and 6B illustrate structural examples of an endoscope apparatus of one embodiment;

FIGS. 7A to 7C illustrate a structural example of an endoscope apparatus of one embodiment;

FIGS. 8A and 8B illustrate a structural example of an endoscope apparatus of one embodiment;

FIGS. 9A to 9C illustrate a structural example of an endoscope apparatus of one embodiment;

FIG. 10 illustrates a structural example of an endoscope apparatus of one embodiment;

FIG. 11 illustrates an example of a method for controlling an endoscope apparatus of one embodiment;

FIGS. 12A to 12C illustrate a structural example of a light-emitting panel of one embodiment;

FIGS. 13A to 13C illustrate structural examples of a light-emitting panel of one embodiment;

FIGS. 14A and 14B illustrate structural examples of a light-emitting panel of one embodiment;

FIGS. 15A to 15D illustrate light-emitting elements;

FIGS. 16A and 16B illustrate structural examples of a light-emitting panel of one embodiment;

FIG. 17 illustrates a structural example of a light-emitting panel of one embodiment;

FIG. 18 shows voltage-luminance characteristics of a light-emitting panel of one example;

FIG. 19 shows an emission spectrum of a light-emitting panel of one example;

FIGS. 20A and 20B illustrate a structural example of a light-emitting panel of one embodiment;

FIG. 21 shows voltage-luminance characteristics of a light-emitting panel of one example;

FIG. 22 shows an emission spectrum of a light-emitting panel of one example;

FIGS. 23A and 23B illustrate a structural example of an endoscope apparatus of one embodiment;

FIGS. 24A and 24B illustrate structural examples of an endoscope apparatus of one embodiment;

FIGS. 25A to 25C illustrate a structural example of an endoscope apparatus of one embodiment; and

FIGS. 26A and 26B illustrate structural examples of an endoscope apparatus of one embodiment.

### DETAILED DESCRIPTION OF THE INVENTION

Embodiments will be described in detail with reference to drawings. Note that the present invention is not limited to the description below, and it is easily understood by those skilled in the art that various changes and modifications can be made without departing from the spirit and scope of the present invention. Accordingly, the present invention should not be interpreted as being limited to the content of the embodiments below.

Note that in the structures of the invention described below, the same portions or portions having similar functions are denoted by the same reference numerals in different drawings, and description of such portions is not repeated. Further, the same hatching pattern is applied to portions having similar functions, and the portions are not especially denoted by reference numerals in some cases.

Note that in each drawing described in this specification, the size, the layer thickness, or the region of each component is exaggerated for clarity in some cases. Therefore, embodiments of the present invention are not limited to such a scale.

### Embodiment 1

In this embodiment, an example of an endoscope apparatus of one embodiment of the present invention is described with reference to drawings.

### Structural Example

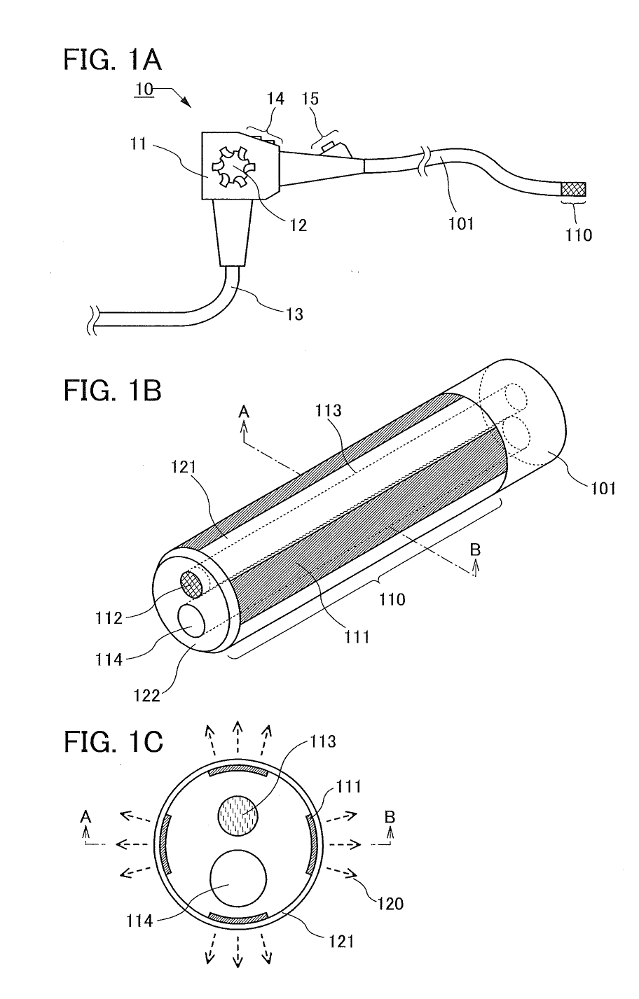

FIG. 1A illustrates an endoscope apparatus 10 exemplified in this embodiment. The endoscope apparatus 10 includes an operation portion 11 that can be gripped. The operation portion 11 is provided with an operation dial 12, an operation button 14, and a forceps opening 15, and the like. In addition, the operation portion 11 is connected to a connector 13 and an insertion portion 101. The insertion portion 101 has an end portion 110 at its end.

The insertion portion 101 is tubular and can be curved. At least part of the end portion 110 (e.g., an end surface 122 described later and its vicinity) of the insertion portion 101 is preferably hard. By operating the operation dial 12 provided in the operation portion 11, a part of the insertion portion 101 is curved and the direction of the end portion 110 can be freely changed.

The operation button 14 provided in the operation portion 11 has a function of, for example, supplying liquid such as water and a gas such as air to a through hole provided in the end portion 110 and operating suction and the like through the through hole. The forceps opening 15 leads to the through hole provided in the end portion 110, and by inserting a forceps or the like to the forceps opening 15, treatment can be performed. Note that a tube (such as a catheter) may be inserted to the forceps opening 15 to perform suction, air supply, water supply, or the like.

The connector 13 connected to the operation portion 11 may be connected to a control device or the like which is not illustrated, and may be provided with a wiring for transmitting an image signal from the end portion 110, a wiring for supplying power to a light-emitting device 111 provided in the end portion 110, a hole for supplying the above liquid and air, and the like. The insertion portion 101 is provided with a hole leading to the forceps opening 15 in addition to the wirings and the hole similar to those of the connector 13.

The endoscope apparatus of one embodiment of the present invention may be used for medical applications to inspect, observe, or treat an inspection object, assuming that the inspection object is a human body. Alternatively, assuming that the inspection object is equipment, machinery, or the like other than the human body, the endoscope apparatus of one embodiment of the present invention may be used for inspection applications to inspect, observe, or repair the inside of these objects.

For example, in the case of using an endoscope apparatus specialized in observing and inspecting, in the operation portion 11, at least the operation dial 12 may be provided, and the operation button 14, the forceps opening 15, and the like are not necessarily provided.

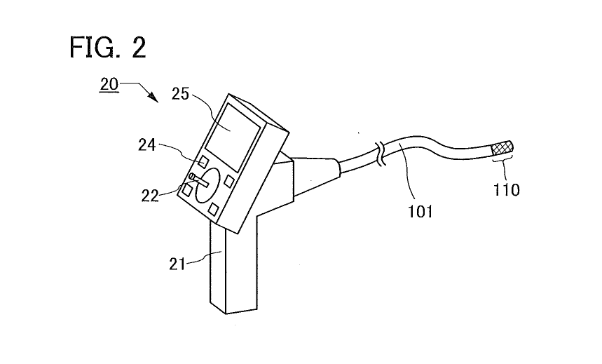

Alternatively, as in an endoscope apparatus 20 illustrated in FIG. 2, an operation portion 21 may be provided with an operation stick 22, an operation button 24, a display portion 25, and the like. The endoscope apparatus 20 does not need to have a control device and the like and thus has high portability. Note that in the endoscope apparatus 20, the operation portion 21 may be mounted with a battery or may be connected to, for example, a cable for supplying power.

### Structural Example of End Portion

Next, a structural example of the end portion 110 of the insertion portion 101 is described.

### Structural Example 1

FIG. 1B is a perspective view of the end portion 110 shown in this structural example. FIG. 1C is a cross-sectional schematic view taken along the line A-B in FIG. 1B.

The end portion 110 includes a tubular exterior member 121. The end surface 122 of the exterior member 121 is provided with an optical device 112, a hole 114, and the like. In addition, inside the exterior member 121, a belt-shaped light-emitting device 111, a transmission path 113 connected to the optical device 112, and the like are provided.

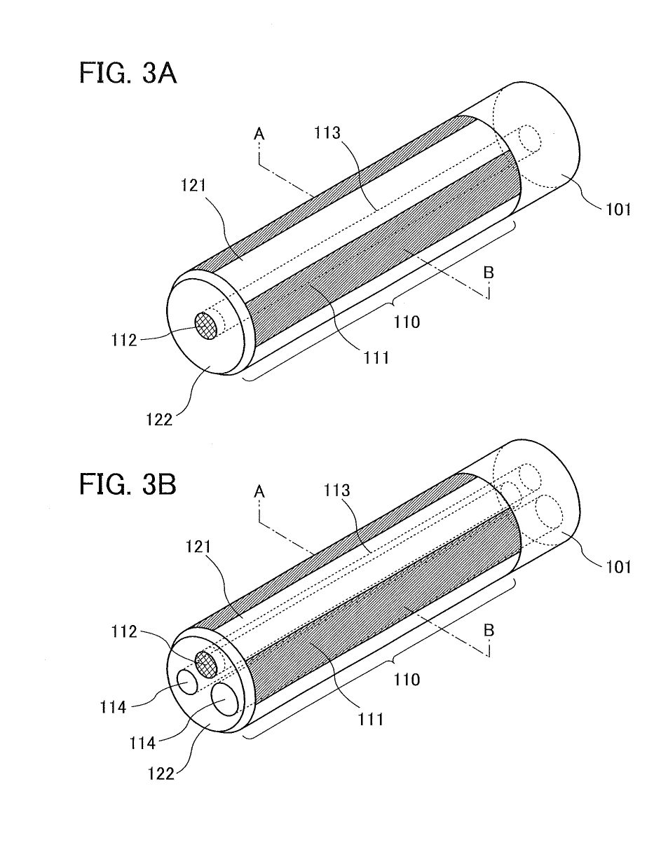

Here, one hole 114 is provided in the end surface 122; however, the hole 114 is not necessarily provided as illustrated in FIG. 3A, or two or more holes 114 may be provided as illustrated in FIG. 3B depending on the purpose. FIG. 3B shows an example in which two holes 114 with different sizes are provided.

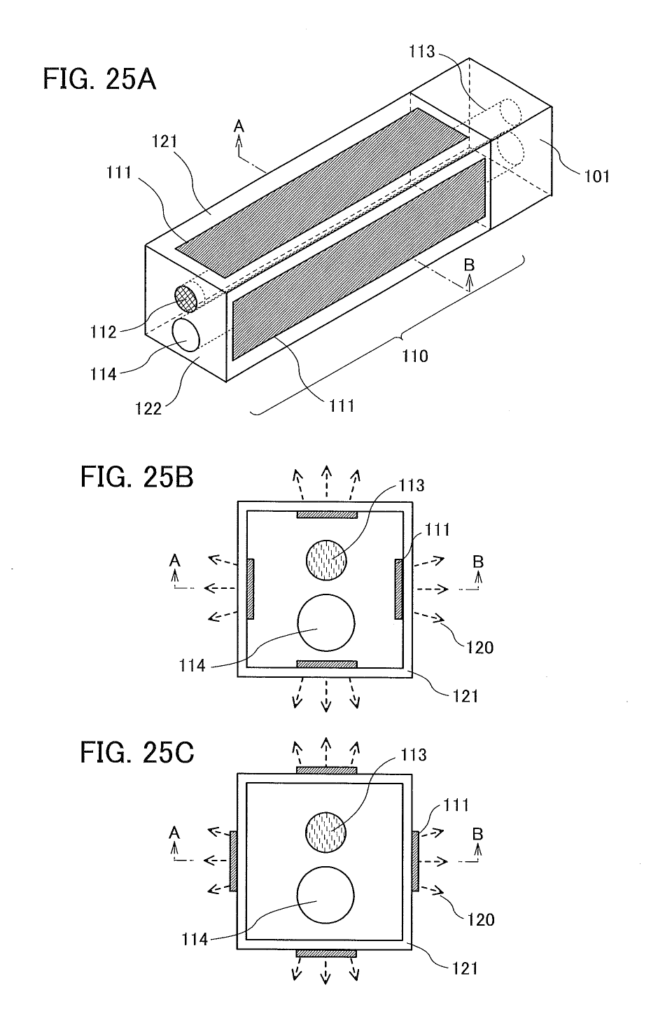

Furthermore, here, the exterior member 121 has a tubular shape; however, the shape is not limited to this, and the exterior member 121 may have a columnar shape or a spindle shape whose cross-sectional shape perpendicular to the extension direction is a polygon, an ellipse, or the like. An example in which the cross-sectional shape perpendicular to the extension direction is a quadrangle is shown in FIGS. 25A, 25B, and 25C.

The light-emitting device 111 has a long belt shape in the extension direction of the exterior member 121 and is provided along the side surface of the exterior member 121. As illustrated in FIG. 1C, light 120 from the light-emitting device 111 is emitted from the inside of the exterior member 121 to the outside.

When the insertion portion 101 is inserted into the inspection object, the light-emitting device 111 can illuminate an inner wall of the inspection object. The light emitted to the inner wall of the inspection object is reflected by the inner wall and scattered, so that the front of the end surface 122 can be indirectly illuminated with the light. At this time, the light illuminating the front is the light reflected by the inner wall (scattered light from the inner wall) around the end portion 110 and is the light with low directivity. As a result, a shadow is less easily produced inside the inspection object as compared to the case where a point light source (such as an LED or an optical fiber) is provided on the end surface 122 to illuminate the front; thus, a region that could not be viewed because of the shadow can be viewed. In particular, in the case where the inside of the inspection object has a shape including a lot of projections and depressions and in the case where the inside of machinery in which many components such as gears are incorporated is inspected, detailed observation in the depth direction can be performed.

Here, the light-emitting device 111 may have a structure in which a plurality of point light sources (such as an LED) is placed in a planar shape along the side surface of the exterior member 121. In particular, a surface light-emitting device is preferably used. When the surface light-emitting device 111 is used, the inner wall of the inspection object can be uniformly irradiated, and the directivity of the light reflected by the inner wall (scattered light from the inner wall) can be further reduced.

As the light-emitting device 111, a light-emitting device including a light-emitting element containing a light-emitting organic compound (organic electroluminescence (EL) element) is preferably used. In particular, a light-emitting device including a light-emitting element which emits white light is preferably used. The organic EL element emitting white light has a higher color rendering property than the LED and the like; thus, it is possible to detect a slight difference in color inside the inspection object. Furthermore, with the organic EL element, the color rendering property on the long wavelength side can be increased as compared to the case with the LED or the like. For example, a difference in color between the normal tissue and the degenerated tissue (cancer cell) inside a living body is slight; thus, it is difficult to detect the degenerated portion, particularly at an early stage, in the case where the LED or the like is used. However, by using the light-emitting device 111 including an organic EL element with high color rendering property on the long wavelength side, the degenerated portion can be easily detected at an early stage.

Note that one embodiment of the present invention is not limited thereto. Another light source of the organic EL element may be additionally provided in some cases. Alternatively, the LED may be provided in addition to the organic EL element.

Furthermore, as the light-emitting device 111, a flexible light-emitting panel is preferably used. For example, an extremely thin light-emitting panel in which a light-emitting element is provided over a flexible substrate is used. As described above, the light-emitting device 111 has flexibility, whereby the light-emitting device 111 can be easily deformed along an inner curved surface of the exterior member 121. Moreover, in the case where a flexible material such as an elastic body is used as the exterior member 121, even when the exterior member 121 is curved or the exterior member 121 is deformed by external force, the light-emitting device 111 can be deformed in accordance with the deformation of the exterior member 121. For example, the insertion portion 101 is inserted into the inspection object and the end portion 110 is easily deformed when the end portion 110 is in contact with the inner wall of the inspection object, whereby the inner wall of the inspection object can be prevented from being damaged, for example.

Note that one embodiment of the present invention is not limited thereto. For example, a light-emitting panel provided over a glass substrate may be provided in addition to the flexible light-emitting panel or instead of the flexible light-emitting panel.

In FIGS. 1A to 1C, four light-emitting devices 111 are provided along the extension direction of the exterior member 121; however, the number of the light-emitting devices 111 is not limited to this and one or more light-emitting devices 111 may be provided. In the case where the plurality of light-emitting devices 111 is provided, the light-emitting devices 111 are preferably arranged at regular intervals along the periphery of the exterior member 121 to improve the uniformity of light. Furthermore, in the case where the plurality of light-emitting devices 111 is provided, these light-emitting devices 111 may emit light with the same luminance or emission luminance may be individually controlled. For example, emission luminance of the plurality of light-emitting devices 111 is individually controlled in accordance with a positional relationship or a distance between the end portion 110 and the inner wall of the inspection object, whereby the front of the end surface 122 can have optimal brightness.

In the exterior member 121, for at least a region to which light from the light-emitting device 111 is emitted, a transparent member is used. To increase the light extraction efficiency, at least one of the inside and the outside of the exterior member 121 may be provided with a light extraction member such as a microlens array or a light diffusion sheet, or a surface of the exterior member 121 may be provided with fine unevenness.

As the optical device 112, for example, an image sensor such as a CCD image sensor and a complementary metal oxide semiconductor (CMOS) image sensor, a fiberscope including an optical fiber, or the like can be used. In particular, from the viewpoint of the resolution and color reproducibility, the CCD image sensor is preferably used.

In the case were the image sensor is used as the optical device 112, a signal line connected to the image sensor and a wiring such as a power supply line correspond to the transmission path 113. Alternatively, in the case where the fiberscope is used as the optical device 112, the transmission path 113 and the optical device 112 are not distinguished, and both of them correspond to a bundle of optical fibers.

One embodiment of the present invention is not limited thereto, and for example, the transmission path 113 is not provided, and data may be wirelessly transmitted instead.

In the endoscope apparatus 10 including the insertion portion 101 including the end portion 110 shown in the structural example, the end portion 110 is provided with a light source that illuminates the inside of the inspection object, whereby it is not necessary that a relatively large light source be provided outside as in the case where the xenon lamp is used as the light source. Thus, the structure of the endoscope apparatus can be simplified. Moreover, light is emitted from the side surface of the end portion 110 of the insertion portion 101 to illuminate the inner wall of the inspection object, whereby the shadow is not easily produced, and an endoscope apparatus which can observe the inside of the inspection object in detail can be obtained. In particular, the light-emitting device 111 in which an organic EL element is used in the end portion 110 is used, whereby an endoscope that can detect a slight difference in color can be obtained.

The above is the description of Structural Example 1.

### Modification Example 1

A structural example of the end portion 110 that is partly different from Structural Example 1 is described below. Note that description of the portions already described is omitted and different portions are described in detail.

In Structural Example 1, the belt-shaped light-emitting device 111 is provided along the extension direction of the exterior member 121; however, one embodiment of the present invention is not limited thereto, and the light-emitting device 111 can be provided as follows, for example.

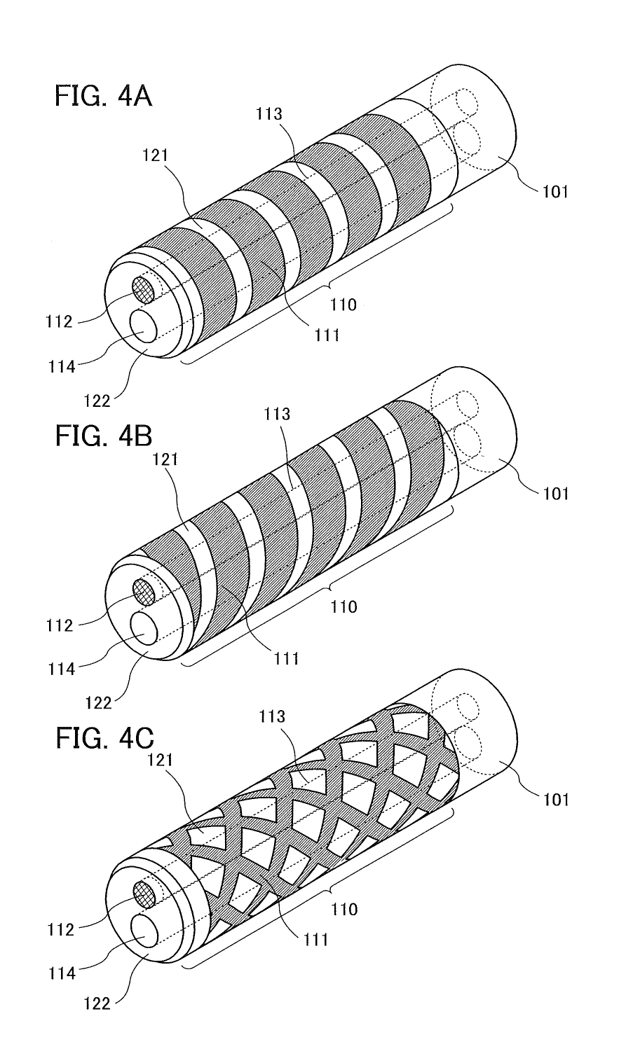

In a structure in FIG. 4A, the belt-shaped light-emitting device 111 is provided along the circumference direction of the side surface of the tubular exterior member 121. In FIG. 4A, the plurality of belt-shaped light-emitting devices 111 is provided; however, the number of the belt-shaped light-emitting devices 111 is preferably one or more. By providing the plurality of light-emitting devices 111 in parallel, a light-emitting area can be increased. By providing the plurality of belt-shaped light-emitting devices 111 which is apart from each other, stress applied to the light-emitting device 111 can be relieved even when the end portion 110 is curved, so that the light-emitting device 111 can be prevented from being damaged.

As describe above, the plurality of light-emitting devices 111 may be made to emit light at the same luminance, or the emission luminance of the plurality of light-emitting devices 111 may be individually controlled.

In a structure in FIG. 4B, the belt-shaped light-emitting device 111 is provided in a spiral along the side surface of the tubular exterior member 121. With such a structure, as in the structure in FIG. 4A, stress applied to the light-emitting device 111 because of the deformation of the end portion 110 can be relieved. Moreover, one long belt-shaped light-emitting device is placed in a spiral in this manner, whereby the number of components can be reduced.

In FIG. 4C, a light-emitting device 111 with a net-like shape is provided along the side surface of the exterior member 121. The light-emitting device 111 has a net-like shape in this manner, whereby in-plane uniformity in emission of the light-emitting device 111 can be improved and the light-emitting device 111 can have elasticity in the extension direction of the end portion 110; thus, stress applied to the exterior member 121 at the time of deformation of the exterior member 121 can be reduced.

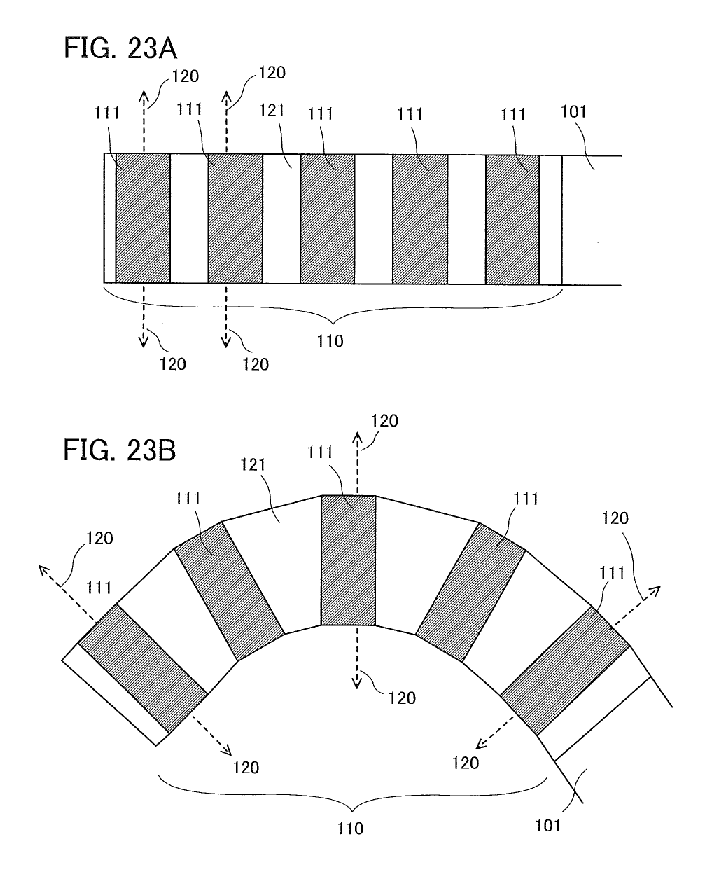

With the use of the flexible exterior member 121, the end portion 110 may have flexibility. FIG. 23A is a side view of the end portion 110 in FIG. 4A. FIG. 23B illustrates the end portion 110 part of which is curved. It is preferable that the state of FIG. 23A be freely changed into the state of FIG. 23B. As a result, the emission direction of the light 120 can be changed. In other words, the light-emission direction of a light source can be controlled. Consequently, the light 120 can be emitted from an appropriate direction; thus, a shadow cannot be easily produced.

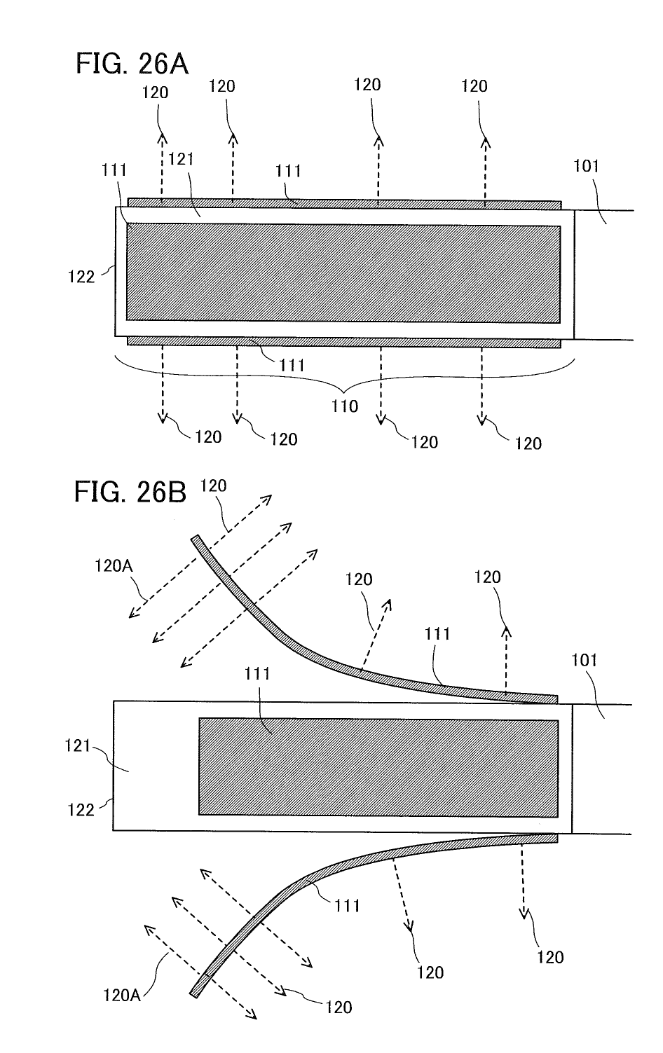

Alternatively, it is preferable that only the light-emitting device 111 be freely deformed. For example, FIG. 26A is a side view of the end portion 110 in FIG. 25A. Here, FIG. 26B illustrates an example in which only the light-emitting device 111 is deformed. The light-emitting device 111 in a portion that is far from the end surface 122 is supported and the light-emitting device 111 in a portion near the top surface 122 is curved toward the outside of the exterior member 121 to be apart from the exterior member 121, whereby the light-emitting device 111 is deformed so that a rear surface (a surface in contact with the exterior member 121 before the deformation) of the light-emitting device 111 can be seen from the top surface 122 side. The curvature of the portion in which the light-emitting device 111 is curved toward the outside of the exterior member 121 can be changed as needed. As a result, only the light-emitting device 111 can be deformed while the exterior member 121 and the optical device 112 in the exterior member 121 are not deformed. Thus, only the emission direction of the light 120 can be changed. Consequently, for example, when there is a projection or the like inside the inspection object, only the emission direction of the light 120 can be changed while the position of the optical device 112 is not changed; thus, unevenness, depressions and projections, and the like can be observed in more detail. In other words, a three-dimensional observation can be easily achieved.

In the case where the light-emitting device 111 is moved, a dual-emission light-emitting device is preferably used as the light-emitting device 111. Alternatively, light is emitted from both sides because two light-emitting devices 111 overlap with each other, which is preferable. As a result, as illustrated in FIG. 26B, not only the light 120, but also a light 120A is emitted; thus, the state of lighting can be controlled more appropriately. In the case where the light 120A is emitted, the light 120 is not necessarily emitted.

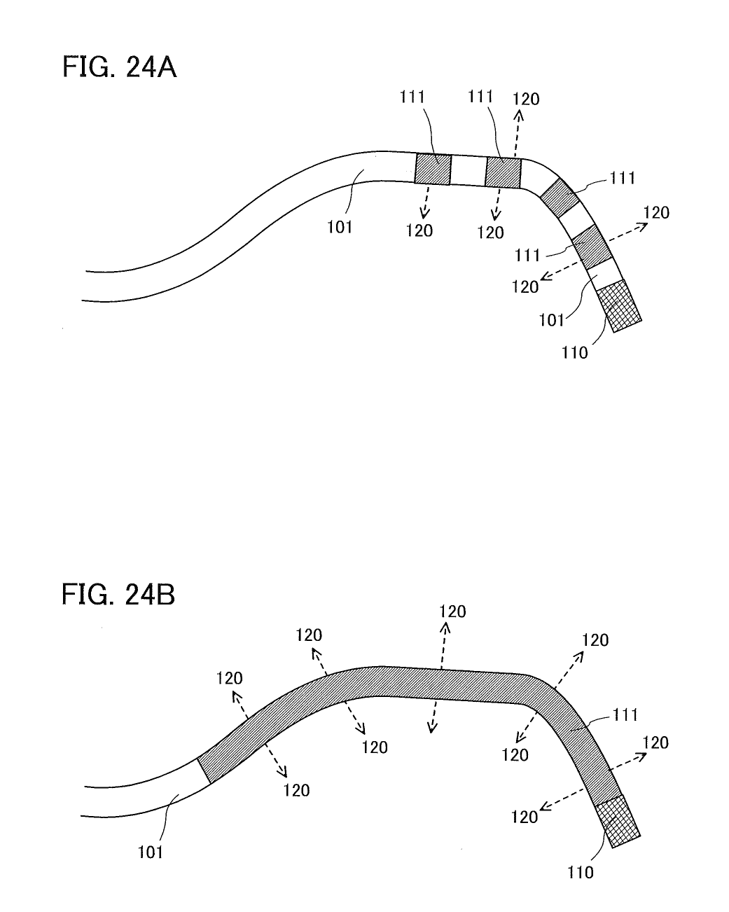

An example in which the light-emitting device 111 is provided in the end portion 110; however, one embodiment of the present invention is not limited thereto. The light-emitting device 111 may be provided in only a position other than the end portion 110 or may be provided in both of the end portion 110 and a position other than the end portion 110. In FIGS. 24A and 24B, examples in which the light-emitting device 111 is provided in a region other than the end portion 110 in the insertion portion 101 are shown. The light-emitting device 111 is provided in the insertion portion 101 near the end portion 110, whereby a shadow cannot be easily produced. Moreover, in the case where the insertion portion 101 has flexibility, the emission direction of the light 120 can be changed by the flexible light-emitting device 111. In other words, the light-emission direction of the light source can be controlled. As a result, the light 120 can be emitted from an appropriate direction; thus, a shadow cannot be easily produced.

The above is the description of Modification Example.

### Structural Example 2

A structural example of the end portion 110 that can also emit light to the front of the end surface of the end portion 110 as well as from the side surface of the end portion 110.

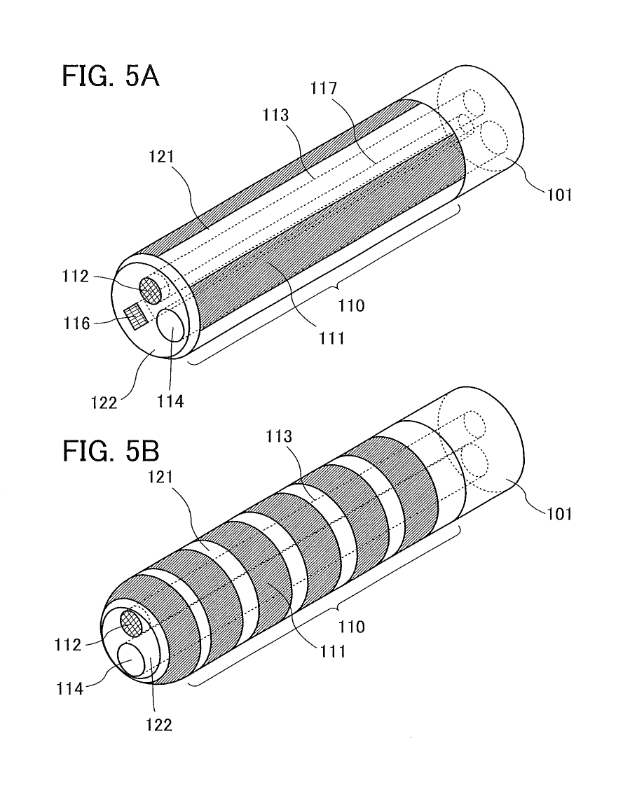

In the end portion 110 in FIG. 5A, in addition to the structure in FIGS. 1A to 1C, the side surface 122 is provided with a light-emitting portion 116 and a transmission path 117 connected to the light-emitting portion 116.

As the light-emitting portion 116, a light-emitting element such as an LED element and an organic EL element, an optical fiber that transmits light from a light source such as a xenon lamp, or the like can be used. In particular, the organic EL element is preferably used in terms of a color rendering property of light and simplification of an apparatus.

In the case where the light-emitting element is used as the light-emitting portion 116, the transmission path 117 corresponds to a wiring that transmits signals and power for driving the light-emitting element. On the other hand, in the case where the optical fiber or the like is used as the light-emitting portion 116, the light-emitting portion 116 and the transmission path 117 are not distinguished from each other and both of them correspond to the optical fiber.

As described above, by providing the light-emitting device 111 that emits light from the side surface of the end portion 110 and the light-emitting portion 116 that emits light to the front of the end surface 122 at the same time, the front of the end surface 122 can be brightly irradiated with the light from the light-emitting portion 116 and the shadow portion that can be produced at this time can be irradiated with light reflected by an inner wall of an inspection object; thus, the inside of the inspection object can be observed in detail.

A structure of the end portion 110 in FIG. 5B is different from the structure in FIG. 4A in the shape of the end portion 110 around the end surface 122. Specifically, the external diameter of the end surface 122 is smaller than the external diameter of the exterior member 121, a surface shape has a gentle gradient from the end surface 122 to the side surface of the exterior member 121, and the light-emitting device 111 is placed along the gradient portion. Thus, part of the light from the light-emitting device 111 is emitted to the front of the end surface 122; thus, the front of the end surface 122 can be brightly irradiated.

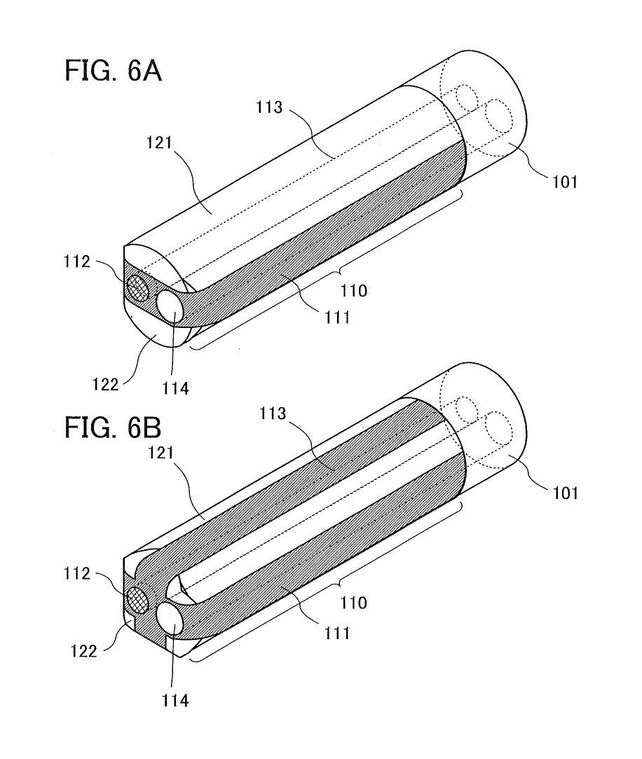

In a structure of the end portion 110 in FIG. 6A, a belt-shaped light-emitting device 111 is provided to cover the side surface of the exterior member 121 and part of the end surface 122. In a structure of the end portion 110 in FIG. 6B, a cross-shaped light-emitting device 111 is provided to cover the side surface of the exterior member 121 and part of the end surface 122. Furthermore, in the structures in FIGS. 6A and 6B, openings are formed in the light-emitting device 111 in regions overlapping with the hole 114 and the optical device 112 provided on the end surface 122.

As described above, by providing the light-emitting device 111 to cover part of the end surface 122, light can be emitted from the side surface of the end portion 110 and to the front of the end surface 122 at the same time, and the front of the end surface 122 can be brightly irradiated and the shadow portion that can be produced at this time can be irradiated with light reflected by the inner wall of the inspection object; thus, the inside of the inspection object can be observed in detail.

In addition, as illustrated in FIGS. 6A and 6B, one continuous light-emitting device 111 is provided to cover the side surface of the exterior member 121 and part of the end surface 122, whereby the number of components can be reduced.

The above is the description of Structural Example 2.

### Structural Example 3

A structural example of the end portion 110 whose structure is partly different from the above structure is described below.

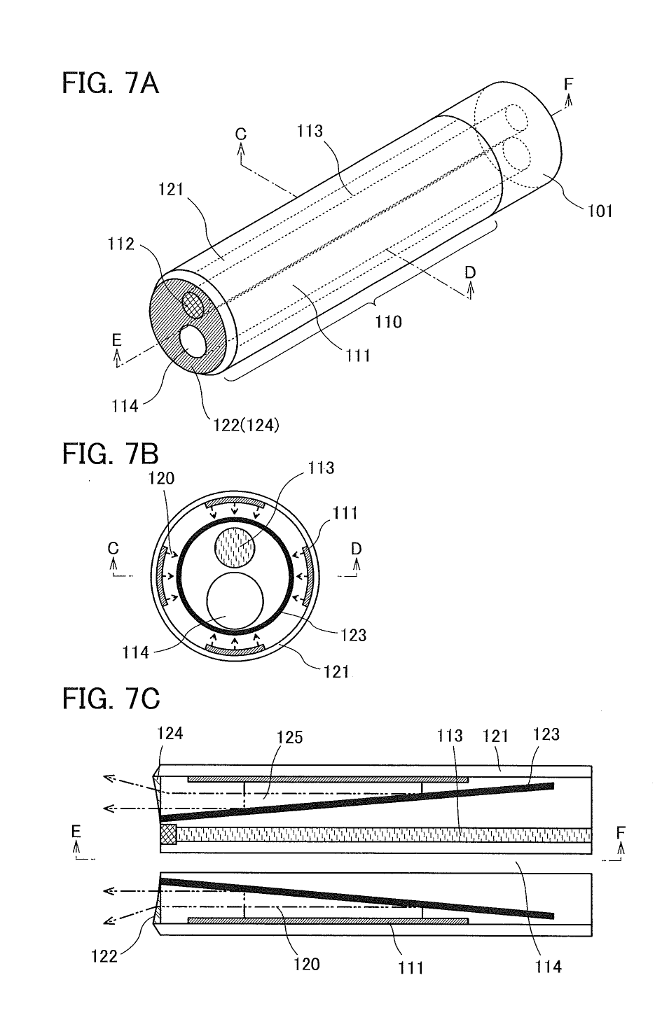

FIG. 7A is a schematic perspective view of the end portion 110 described in this structural example. FIGS. 7B and 7C are schematic cross-sectional views taken along the section lines C-D and E-F in FIG. 7A, respectively.

The end portion 110 in FIGS. 7A to 7C is mainly different from the structure of the end portion in FIGS. 1A to 1C in the structure of the end surface 122 and the light-emitting direction of the light-emitting device 111, and in that a reflection member 123 is provided.

The light-emitting device 111 is provided along the side surface of the exterior member 121 and light is emitted from the side surface of the exterior member 121 to the inside thereof.

The reflection member 123 provided inside the exterior member 121 reflects light at, at least, a surface facing the light-emitting device 111. As illustrated in FIG. 7C, the reflection member 123 is placed to be inclined to the extension direction of the end portion 110 so that the distance between the reflection member 123 and the light-emitting device 111 becomes longer as the reflection member 123 is close to the end surface 122. Thus, the light 120 emitted from the light-emitting device 111 is reflected by the reflection member 123, and part of the light 120 is emitted to the front of the end surface 122.

It is preferable that the angle between the reflection member 123 and the light-emitting device 111 be closer to 45′; however, as the angle between the reflection member 123 and the light-emitting device 111 is smaller, a light-emitting area of the light-emitting device 111 can be increased and the luminance of the light emitted from the end surface 122 can be higher. The angle between the reflection member 123 and the light-emitting device 111 may be set as appropriate in the range of 1° to 45° in consideration of an internal diameter of the exterior member 121, thicknesses of the light-emitting device 111, the reflection member 123, and the like, and a spatial limitation.

A region 125 between the reflection member 123 and the light-emitting device 111 is filled with a light-transmitting material. Here, when a material that has a high index of refraction is used for the region 125 between the reflection member 123 and the light-emitting device 111, total reflection easily occurs between the light-emitting device 111 and the region 125, and the reflection is repeated between the reflection member 123 and the light-emitting device 111, whereby the light can be confined in the region 125. As a result, the luminance of the light emitted from the end surface 122 can be increased.

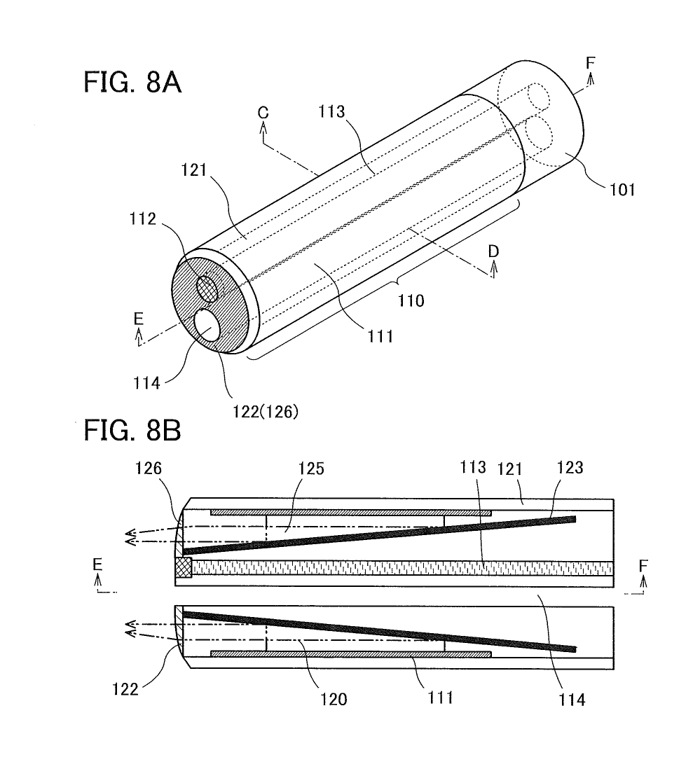

On the end surface 122 in a region from which light is emitted, a concave lens 124 is provided. With the concave lens 124, the light emitted from the end surface 122 can be diffused; thus, the front of the end surface 122 can be observed over a wider range. Alternatively, as illustrated in FIGS. 8A and 8B, instead of the concave lens 124, a convex lens 126 may be provided. By placing the convex lens 126 on the end surface 122, the light emitted from the end surface 122 can converge to increase the luminance.

A Fresnel lens is used as the concave lens 124 and the convex lens 126, whereby the thickness of the lens can be reduced. Alternatively, instead of the concave lens 124 or the convex lens 126, a cylindrical lens, a toroidal lens, or a light extraction member such as a microlens array or a light diffusion sheet may be provided on the end surface 122. By providing the lens or the light extraction member, light extraction efficiency can be improved to increase the luminance of the light emitted from the end surface. Note that such a lens and a light extraction member are not necessarily provided.

In the structures of the end portion 110 in FIGS. 7A to 7C and FIGS. 8A and 8B, almost all the region of the end surface 122 other than the region where the optical device 112 and the hole 114 are provided can be used as a light-emitting region. In other words, surface light emission can be performed to the front of the end surface 122. Thus, as compared to the case where the point light source (such as an LED and an optical fiber) is provided on the end surface 122 and illuminates the front, a shadow is not easily produced inside the inspection object; thus, the inside of the inspection object can be observed in detail.

In addition, by using a surface light-emitting device as the light-emitting device 111 provided in the exterior member 121, the light-emitting area of the light-emitting device 111 is extremely large as compared to the case of using the point light source such as an LED. Thus, the light from the surface light-emitting device 111 converges and is emitted from the end surface 122, whereby light with extremely high luminance can be emitted to the front of the end surface 122.

### Modification Example 2

A structural example of the end portion 110 that is partly different from Structural Example 3 is described below.

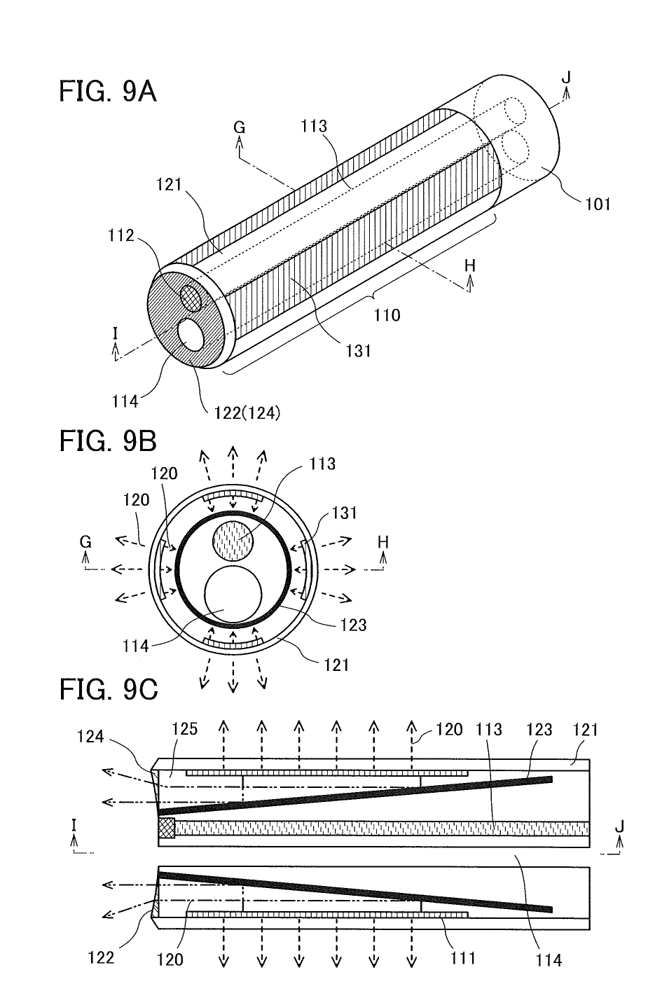

FIG. 9A is a schematic perspective view of the end portion 110 described below. FIGS. 9B and 9C are schematic cross-sectional views taken along the section lines G-H and I-J in FIG. 9A, respectively.

The end portion 110 in FIGS. 9A to 9C is mainly different from Structural Example 3 in that a light-emitting device 131 is provided instead of the light-emitting device 111. The light-emitting device 131 is a dual-emission light-emitting device.

As illustrated in FIGS. 9B and 9C, the light-emitting device 131 emits the light 120 from the side surface of the exterior member 121 to the inside thereof and emits the light 120 from the side surface of the exterior member 121 to the outside thereof.

The light 120 emitted from the side surface of the exterior member 121 to the inside thereof is reflected by the reflection member 123, and part of the light 120 is emitted to the front of the end surface 122. In addition, with the light 120 emitted from the side surface of the exterior member 121 to the outside thereof, the inner wall of the inspection object can be irradiated.

With the end portion 110 having such a structure, the front of the end portion 110 can be brightly irradiated, and at the same time, the shadow portion produced by the irradiation can be irradiated with indirect light reflected by the inner wall of the inspection object; thus, the inside of the inspection object can be observed in detail.

The above is the description of the structural example of the end portion.

In each of the structures of the end portions in Structural Example 2, Structural Example 3, and Modification Example 2, the shape of the light-emitting device provided on the side surface of the end portion can be combined with any of the shapes of the light-emitting device in Structural Example 1 and Modification Example 1. In the structural examples and the modification examples, a light-emitting element such as an LED element and an organic EL element, an optical fiber that transmits light from a light source such as a xenon lamp, or the like may be provided on the end surface as appropriate.

### Example of Control Method

A structural example and an example of a control method to control the endoscope apparatus described in the above structural examples and the like are described below.

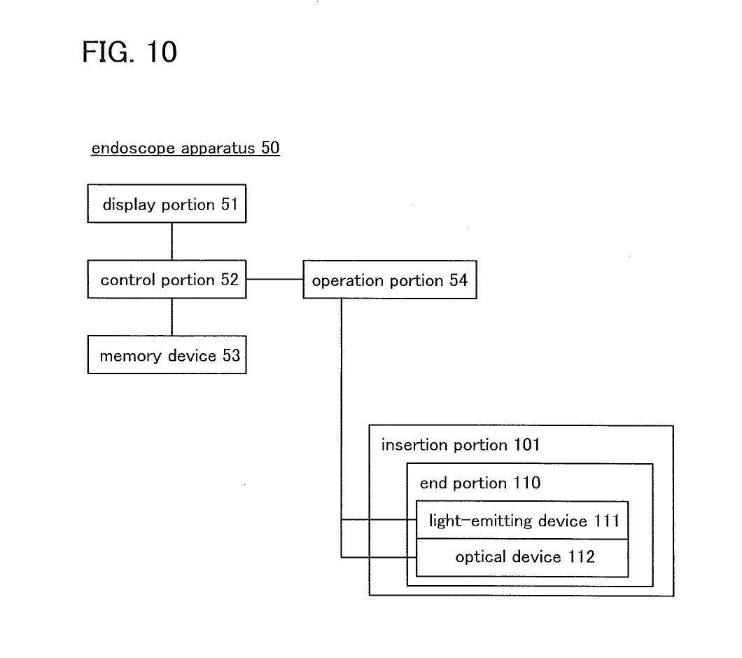

FIG. 10 is a block diagram showing a main part of an endoscope apparatus 50 described below. The endoscope apparatus 50 includes a display portion 51, a control portion 52, a memory device 53, an operation portion 54, and an insertion portion 101. The insertion portion 101 includes an end portion 110 provided with a light-emitting device 111 and an optical device 112.

The insertion portion 101, the end portion 110, the light-emitting device 111, the optical device 112, and the like can have the above structures as appropriate.

The control portion 52 is electrically connected to the light-emitting device 111 and the optical device 112 through a wiring provided in the operation portion 54 and the insertion portion 101, and can control the driving of these devices. For example, the emission luminance, emission time, timing, and the like of the light-emitting device 111 are controlled. In addition, the control portion 52 can drive the optical device 112 in accordance with photographing conditions (e.g., shutter speed, diaphragm value, and focus) and make the optical device 112 take a moving image or a still image. An image signal taken by the optical device 112 is transmitted to the control portion 52. The control portion 52 may control the emission luminance of the light-emitting device 111 appropriately based on an image taken by the optical device 112.

The control portion 52 converts the image signal input from the optical device 112 into a signal for displaying an image on the display portion 51, and the image can be displayed on the display portion 51. In addition, the image is converted into data and the data can be stored in the memory device 53.

Here, in the display portion 51, the pixel preferably includes a light-emitting element containing a light-emitting organic material (an organic EL element). The display portion 51 in which the organic EL element is included in the pixel has high color reproducibility and high contrast; thus, the image taken by the optical device 112 can be displayed with high reproducibility. In particular, the display portion 51 preferably has high resolution such as MD (1920×1080), 4K2K (3840×2048 or 4096×2180), or 8K4K (7680×4320).

In particular, in the case where the light-emitting device 111 provided in the end portion 110 also includes an organic EL element similar to the above organic EL element, the image taken by emission from the organic EL element is displayed on the display portion 51 including the pixel provided with the above organic EL element, whereby the taken image can be displayed with faithful reproducibility. In particular, a slight difference in color that can be detected by the light-emitting device 111 including the organic EL element can be faithfully reproduced on the display portion 51; thus, the light-emitting device 111 can be suitably used for medical or diagnostic use.

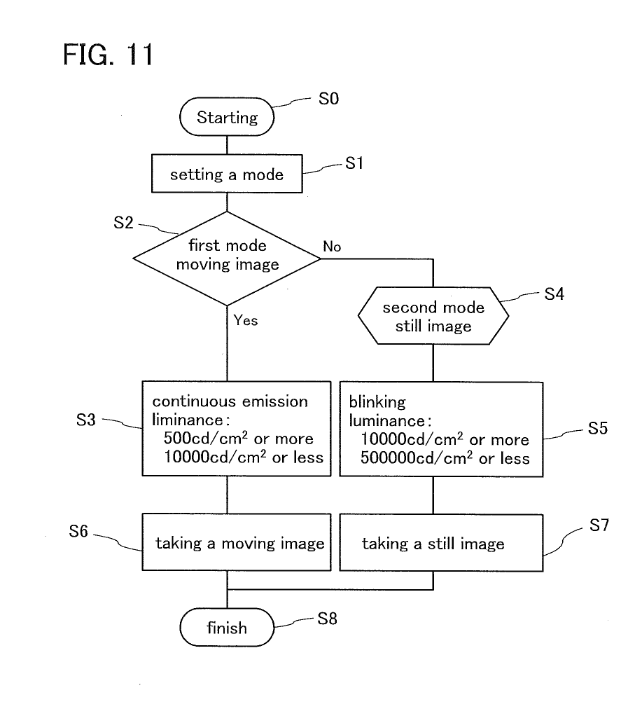

Next, an example of a control operation of the light-emitting device 111 and the optical device 112 in the control portion 52 of the endoscope apparatus 50 is described with reference to FIG. 11. FIG. 11 is a flow chart showing control of the light-emitting device 111 and the optical device 112 in the control portion 52.

First, a photographing operation is started (Step S0).

Next, a mode is set (Step S1). Here, a first mode to observe or take a moving image or a second mode to take a still image is selected. The mode can be set by a user with, for example, a user interface as needed.

In Step S2, when the first mode is selected, the operation proceeds to Step S3. When the mode is not the first mode, that is, the second mode is selected, the operation proceeds to Step S4.

In Step S3, the control portion 52 controls the light-emitting device 111 to emit light with an emission luminance of greater than or equal to 100 cd/cm² and less than 10000 cd/cm², preferably greater than or equal to 500 cd/cm² and less than 10000 cd/cm² continuously or to emit the light intermittently (blinking continuously) so that a flicker in an image is not observed from the light-emitting device 111. After that, the operation proceeds to Step S6. Here, in the case where the light is intermittently emitted, it is preferable that the light be emitted at a frequency of 30 Hz or more, 60 Hz or more, or 120 Hz or more because a flicker in an image is not observed.

By Step S4, the second mode is defined, and the operation proceeds to Step S5. In Step S5, the control portion 52 controls the light-emitting device 111 to emit light with an emission luminance of greater than or equal to 10000 cd/cm² and less than or equal to 500000 cd/cm² by being synchronized with shutter speed of the optical device 112. After that, the operation proceeds to Step S6.

In Step S6, a moving image is taken. At this time, the light with an emission luminance of greater than or equal to 100 cd/cm² and less than 10000 cd/cm², preferably greater than or equal to 500 cd/cm² and less than 10000 cd/cm² is continuously emitted from the light-emitting device 111, which is set in Step S3. Alternatively, the light is intermittently emitted.

In Step S7, a still image is taken. At this time, the light with an emission luminance of greater than or equal to 10000 cd/cm² and less than or equal to 500000 cd/cm² is emitted from the light-emitting device 111 by being synchronized with shutter speed of the optical device 112. For example, by being synchronized with shutter speed of the optical device 112, pulsed light of 1/10000 second or more and 10 seconds or less is emitted from the light-emitting device 111.

In Step S6 or Step S7, at a stage where photographing is terminated, a terminating process is performed (Step S8).

As described above, the luminance and the emission mode of the light-emitting device 111 are preferably changed between when a moving image is taken and when a still image is taken. At the time of taking the moving image, by performing continuous emission in which emission luminance is reduced, deterioration of the light-emitting element of the light-emitting device 111 is suppressed and heat generation from the light-emitting device 111 is suppressed, so that a load of the inspection object can be lightened and observation for a long time can be easily performed. Furthermore, at the time of taking the still image, the luminance of the light-emitting device 111 is increased, whereby a still image having higher image quality can be obtained. In particular, in the case where the emission luminance is low, the resolution needs to be decreased to increase the sensitivity of the optical device 112; however, in the case where the luminance of emission from the light-emitting device 111 is extremely high as described above, the resolution does not need to be decreased to increase the sensitivity, and a still image with higher resolution can be obtained.

The above is the description of the example of the control method.

At least part of this embodiment can be implemented in combination with any of the embodiments described in this specification as appropriate.

### Embodiment 2

In this embodiment, a structure of a light-emitting panel that can be used for the above light-emitting device will be described with reference to drawings.

In view of the above, a light-emitting element that is a planar light source is used for a light-emitting panel in one embodiment of the present invention. For example, with the use of an organic EL element, a thin and large-area element can be formed easily. When a planar light source, a point light source, and a line light source emit the same amount of light, the planar light source can have a smaller amount of light per unit area or a shorter emission time than the point light source and the line light source. Thus, the amount of heat generation per unit area can be reduced. In addition, the planar light source releases heat easily because of its large light-emitting area. Thus, deterioration due to local heat generation of the light-emitting panel can be suppressed. A light-emitting device that has higher reliability and less deterioration of a light-emitting panel than a light-emitting device including a light-emitting diode using an inorganic material, or the like can be provided.

The light-emitting panel can be thinner and lighter in the case of using an organic EL element than in the case of using a conventional xenon lamp or the like. Heat generated by light emission is diffused over a large area in the light-emitting panel and is therefore released efficiently. Thus, heat accumulation in the light-emitting panel is suppressed; and, deterioration of the light-emitting panel is suppressed.

The light-emitting panel can be configured to emit white light by using a properly selected light-emitting organic compound. For example, a plurality of light-emitting organic compounds that emit light of complementary colors can be used. Alternatively, light-emitting organic compounds that emit light of red, green, and blue can be used. Furthermore, different emission spectra can be selected from a variety of organic compounds. Accordingly, the light-emitting device having excellent white balance can be obtained.

By using a light-emitting organic compound, an emission spectrum can be broadened as compared to that of a light-emitting diode with an inorganic material. Light having a broad emission spectrum is close to natural light and suitable for photography.

An example of a structure of a light-emitting panel in which an organic EL element is used as a light-emitting element is described below.

### Structural Example 1

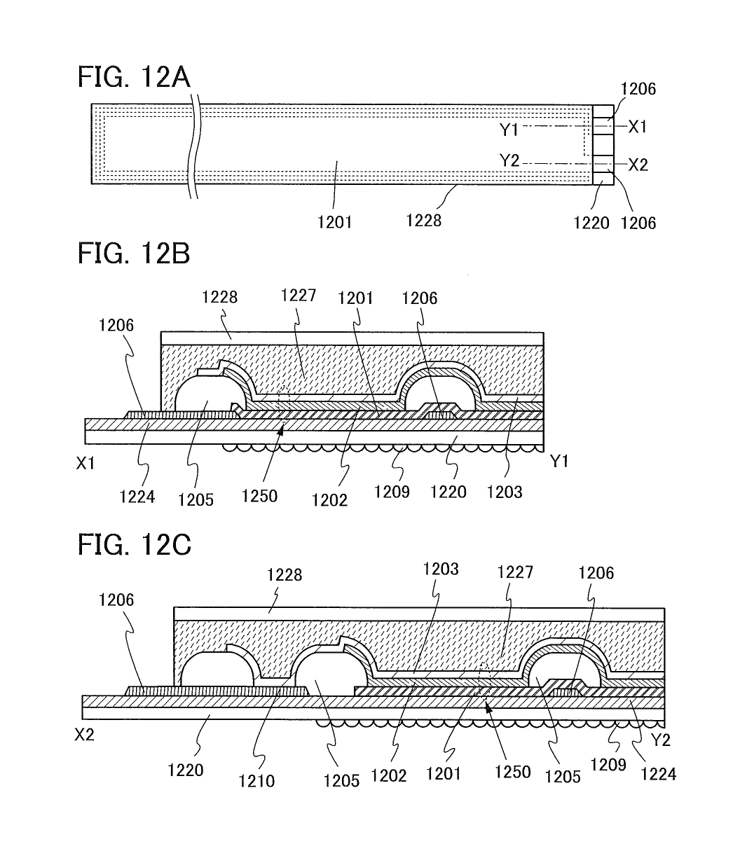

FIG. 12A is a plan view of a light-emitting panel of one embodiment of the present invention, and FIGS. 12B and 12C are cross-sectional schematic views taken along the lines X1-Y1 and X2-Y2 in FIG. 12A, respectively. Note that some components (e.g., a partition wall 1205) are not illustrated in FIG. 12A for simplicity.

In the light-emitting panel illustrated in FIGS. 12A to 12C, a light-emitting element 1250 is provided over a support substrate 1220 with an insulating film 1224 therebetween. The auxiliary wiring 1206 is provided over the insulating film 1224 and is electrically connected to the first electrode 1201. The auxiliary wiring 1206 is partly exposed and functions as a terminal. The conductive layer 1210 is electrically connected to the second electrode 1203, and is partly exposed and functions as a terminal. An end portion of the first electrode 1201 and an end portion of a conductive layer 1210 are covered with a partition wall 1205. In addition, the partition wall 1205 is provided to cover the auxiliary wiring 1206 with the first electrode 1201 therebetween. The light-emitting element 1250 is sealed with the support substrate 1220, a sealing substrate 1228, and a sealant 1227. An outcoupling structure 1209 is attached to the surface of the support substrate 1220. A flexible light-emitting panel can be obtained by using flexible substrates as the support substrate 1220 and the sealing substrate 1228.

The light-emitting element 1250 is an organic EL element having a bottom-emission structure; specifically, the first electrode 1201 transmitting visible light is provided over the support substrate 1220, an EL layer 1202 is provided over the first electrode 1201, and a second electrode 1203 reflecting visible light is provided over the EL layer 1202.

For the outcoupling structure 1209, a hemispherical lens, a micro lens array, a film provided with an uneven surface structure, a light diffusing film, or the like can be used. For example, the outcoupling structure 1209 can be formed by attaching the lens or film to the support substrate 1220 with an adhesive or the like having substantially the same refractive index as the support substrate 1220 or the lens or film.

As methods for forming a light-emitting element over a flexible substrate in the case of fabricating a flexible light-emitting panel, there are methods such as a first method in which the light-emitting element is directly formed over a flexible substrate, and a second method in which the light-emitting element is formed over a highly heat-resistant substrate (hereinafter referred to as a formation substrate) that is different from a flexible substrate and the light-emitting element is then separated from the formation substrate and transferred to the flexible substrate.

When a substrate that is resistant to heat applied in the process of forming the light-emitting element, such as a glass substrate thin enough to have flexibility, is used, the first method is preferably employed, in which case the process can be simplified.

When the second method is employed, an insulating film with low water permeability or the like that is formed over a formation substrate can be transferred to a flexible substrate. Thus, even when an organic resin with high water permeability and low heat resistance or the like is used as a material of the flexible substrate, a flexible light-emitting panel with high reliability can be fabricated.

### Structural Example 2

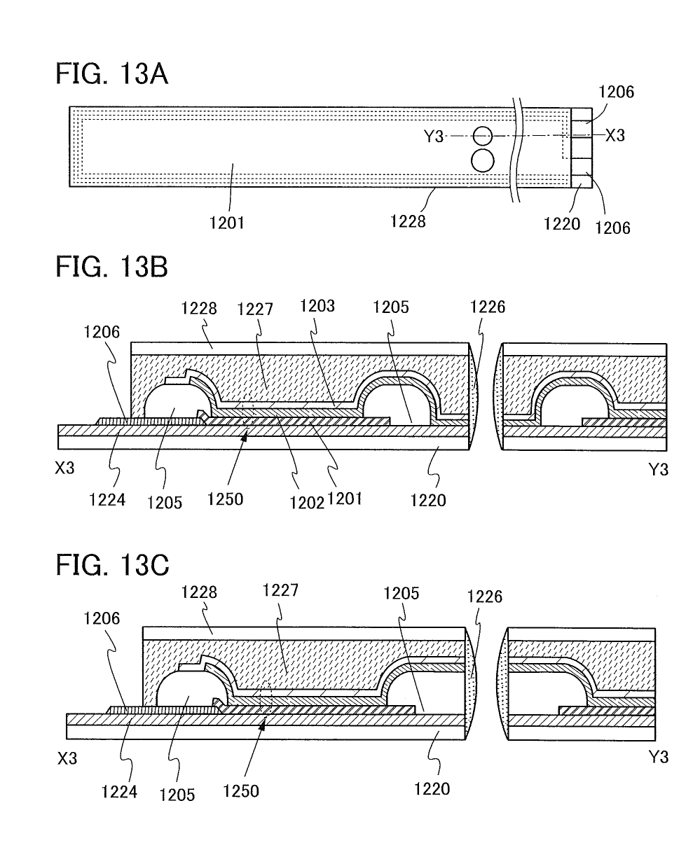

FIG. 13A is a plan view of a light-emitting panel of one embodiment of the present invention, and FIGS. 13B and 13C are cross-sectional schematic views taken along the line X3-Y3 in FIG. 13A. Note that some components (e.g., a partition wall 1205) are not illustrated in FIG. 13A for simplicity.

The light-emitting panel illustrated in FIGS. 13A to 13C is different from the light-emitting panel described in Structural Example 1 in that openings are provided in part of the light-emitting panel. Here, different components are described in detail, and the description of Structural Example 2 can be referred to for the common components.

As illustrated in FIGS. 13B and 13C, the light-emitting panel preferably includes a sealant 1226 in the opening to prevent an electrode or an EL layer from being exposed. Specifically, an opening is formed in the light-emitting panel, and then the sealant 1226 is formed to cover at least an exposed electrode and an exposed EL layer. The sealant 1226 may be the same material as or a different material from the sealant 1227.

FIG. 13B illustrates an example of an opening formed in a region where the partition wall 1205 is not provided. FIG. 13C illustrates an example of an opening formed in a region where the partition wall 1205 is provided.

Note that an outcoupling structure may be provided on a surface of the substrate.

Such a light-emitting panel is manufactured, and the opening is provided at the position overlapping with the optical device 112 and the hole 114 described in Embodiment 1, which can be used in the endoscope apparatus including the end portion 110 exemplified in FIGS. 6A and 6B and the like. Moreover, the shape or the like of the opening is varied, whereby the light-emitting device having a net-like shape as illustrated in FIG. 4C can be obtained.

### Structural Example 3

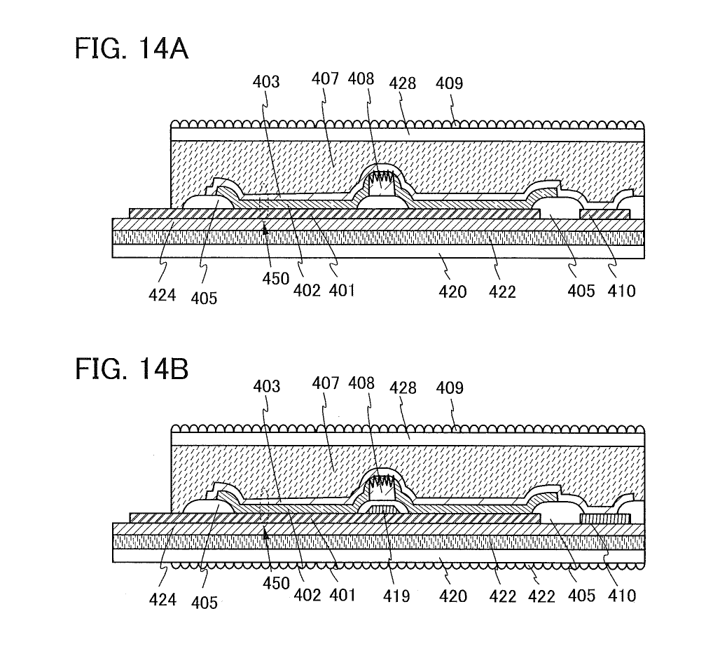

FIG. 14A is a cross-sectional view of a light-emitting panel described below. The light-emitting panel illustrated in FIG. 14A is a top-emission light-emitting panel.

The light-emitting panel illustrated in FIG. 14A includes a flexible substrate 420, an adhesive layer 422, an insulating film 424, a conductive layer 408, an insulating film 405, an organic EL element 450 (a first electrode 401, an EL layer 402, and a second electrode 403), a conductive layer 410, an adhesive layer 407, a flexible substrate 428, and an outcoupling structure 409. The second electrode 403, the adhesive layer 407, the flexible substrate 428, and the outcoupling structure 409 transmit visible light.

The organic EL element 450 is provided over the flexible substrate 420 with the bonding layer 422 and the insulating film 424 provided therebetween. The organic EL element 450 is sealed by the flexible substrate 420, the adhesive layer 407, and the flexible substrate 428. The organic EL element 450 includes the first electrode 401, the EL layer 402 over the first electrode 401, and the second electrode 403 over the EL layer 402. It is preferable that the first electrode 401 reflect visible light. The outcoupling structure 409 is attached to the surface of the flexible substrate 428.

The end portions of the first electrode 401 and the conductive layer 410 are covered with the insulating film 405. The conductive layer 410 can be formed using the same process and material as those of the first electrode 401 and is electrically connected to the second electrode 403.

The conductive layer 408 over the insulating film 405 functions as an auxiliary wiring and is electrically connected to the second electrode 403. Note that the conductive layer 408 may be provided over the second electrode 403. Furthermore, in a manner similar to Structural Example 1, an auxiliary wiring which is electrically connected to the first electrode 401 may be provided.

### Structural Example 4

FIG. 14B is a cross-sectional view of a light-emitting panel described as an example below. The light-emitting panel illustrated in FIG. 14B is a dual-emission light-emitting panel.

The light-emitting panel in FIG. 14B is different from the above Structural Example 3 in that a conductive layer 419 is provided and the outcoupling structure 409 is also provided on the flexible substrate 420. In addition, the first electrode 401, the insulating film 424, the adhesive layer 422, and the flexible substrate 420 transmit visible light.

The conductive layer 419 is provided over the first electrode 401. Moreover, the conductive layer 419 can be formed in the same process and using the same materials as those of the conductive layer 410. The conductive layer 419 serves as an auxiliary wiring and is electrically connected to the first electrode 401. As illustrated in FIG. 14B, the conductive layer 419 is provided to overlap with the insulating film 405, whereby a decrease in light-emitting area can be suppressed, which is preferable.

### [Material of Light-emitting Panel]

Examples of materials that can be used for the light-emitting panel of one embodiment of the present invention are described below.

### [Substrate]

The substrate on the side from which light from the light-emitting element is extracted is formed using a material that transmits the light. For example, a material such as glass, quartz, ceramics, sapphire, or an organic resin can be used.

The weight and thickness of the light-emitting panel can be decreased by using a thin substrate. Furthermore, a flexible light-emitting panel can be obtained by using a substrate that is thin enough to have flexibility.

Examples of glass include alkali-free glass, barium borosilicate glass, and aluminoborosilicate glass.

Examples of a material that has flexibility and transmits visible light include flexible glass, polyester resins such as polyethylene terephthalate (PET) and polyethylene naphthalate (PEN), a polyacrylonitrile resin, a polyimide resin, a poly(methyl methacrylate) resin, a polycarbonate (PC) resin, a polyethersulfone (PES) resin, a polyamide resin, a cycloolefin resin, a polystyrene resin, a polyamide imide resin, and a polyvinyl chloride resin. Specifically, a material whose thermal expansion coefficient is low is preferably used and a polyamide imide resin, a polyimide resin, and polyethylene terephthalate (PET) can be favorably used, for example. A substrate in which a glass fiber is impregnated with an organic resin or a substrate whose thermal expansion coefficient is reduced by mixing an organic resin with an inorganic filler can also be used. A substrate using such a material is lightweight, and a light-emitting panel using this substrate can also be lightweight accordingly.

Since the substrate through which light emission is not extracted does not need to have a light-transmitting property, a metal substrate using a metal material or an alloy material or the like can be used in addition to the above-described substrates. A metal material and an alloy material, which have high thermal conductivity, are preferably used, in which case heat can be conducted to the whole sealing substrate, so that a local temperature rise in the light-emitting panel can be prevented. To obtain flexibility and bendability, the thickness of a metal substrate is preferably greater than or equal to 10 μm and less than or equal to 200 μm, more preferably greater than or equal to 20 μm and less than or equal to 50 μm.

Although there is no particular limitation on a material of the metal substrate, it is preferable to use, for example, aluminum, copper, nickel, a metal alloy such as an aluminum alloy and stainless steel.

It is preferable to use a substrate subjected to insulation treatment in such a manner that a surface of the conductive substrate is oxidized or an insulating film is formed on the surface. An insulating film may be formed by, for example, a coating method such as a spin-coating method or a dipping method, an electrodeposition method, an evaporation method, or a sputtering method. An oxide film may be formed on the substrate surface by exposure to or heating in an oxygen atmosphere or by an anodic oxidation method or the like.

The flexible substrate may have a stacked structure of a layer of any of the above-mentioned materials and a hard coat layer (e.g., a silicon nitride layer) that protects a surface of the light-emitting panel from damage or the like, a layer (e.g., an aramid resin layer) that can disperse pressure, or the like. Furthermore, to suppress a decrease in the lifetime of the light-emitting element due to moisture and the like, an insulating film with low water permeability may be provided. For example, a film containing nitrogen and silicon (e.g., a silicon nitride film, a silicon oxynitride film) or a film containing nitrogen and aluminum (e.g., an aluminum nitride film) may be provided.

The substrate may be formed by stacking a plurality of layers. When a glass layer is used, a barrier property against water and oxygen can be improved and thus a reliable light-emitting panel can be provided.

A substrate in which a glass layer, an adhesive layer, and an organic resin layer are stacked from the side closer to a light-emitting element can be used. The thickness of the glass layer is greater than or equal to 20 μm and less than or equal to 200 μm, preferably greater than or equal to 25 μm and less than or equal to 100 μm. With such a thickness, the glass layer can have both a high barrier property against water and oxygen and a high flexibility. The thickness of the organic resin layer is greater than or equal to 10 μm and less than or equal to 200 μm, preferably greater than or equal to 20 μm and less than or equal to 50 μm. With such an organic resin layer provided on an outer side of the glass layer, breakage or a crack of the glass layer can be inhibited, resulting in increased mechanical strength. With the substrate that includes such a composite material of a glass material and an organic resin, a highly reliable and flexible light-emitting panel can be provided.

### [Insulating Film]

An insulating film may be provided between the support substrate and the light-emitting element. As the insulating film, an inorganic insulating film such as a silicon oxide film, a silicon nitride film, a silicon oxynitride film, and a silicon nitride oxide film can be used. In order to suppress the entrance of moisture or the like into the light-emitting element, an insulating film with low water permeability such as a silicon oxide film, a silicon nitride film, or an aluminum oxide film is particularly preferable. For a similar purpose and with a similar material, an insulating film covering the light-emitting element may be provided.

### [Partition Wall]

For the partition wall, an organic resin or an inorganic insulating material can be used. As the organic resin, for example, a polyimide resin, a polyamide resin, an acrylic resin, a siloxane resin, an epoxy resin, or a phenol resin can be used. As the inorganic insulating material, silicon oxide, silicon oxynitride, or the like can be used. In particular, a photosensitive resin is preferably used for easy formation of the partition wall.

There is no particular limitation on the method for forming the partition wall. A photolithography method, a sputtering method, an evaporation method, a droplet discharging method (e.g., an inkjet method), a printing method (e.g., a screen printing method or an offset printing method), or the like can be used.

### [Auxiliary Wiring]

The auxiliary wiring is not necessarily provided; however, the auxiliary wiring is preferably provided because voltage drop due to the resistance of an electrode can be prevented.

For the auxiliary wiring, a single layer or a stacked layer using a material selected from copper (Cu), titanium (Ti), tantalum (Ta), tungsten (W), molybdenum (Mo), chromium (Cr), neodymium (Nd), scandium (Sc), or nickel (Ni) or an alloy material including any of these materials as its main component is used. Aluminum can also be used as a material of the auxiliary wiring; in this case, in order to prevent corrosion, it is preferable that the auxiliary wiring have a stacked-layer structure and aluminum be used for a layer that is not in contact with ITO or the like. The thickness of the auxiliary wiring can be greater than or equal to 0.1 μm and less than or equal to 3 μm, preferably greater than or equal to 0.1 μm and less than or equal to 0.5 μM

### [Sealant]

A method for sealing the light-emitting panel is not limited, and either solid sealing or hollow sealing can be employed. For example, a glass material such as a glass frit, or a resin material such as a resin that is curable at room temperature (e.g., a two-component-mixture-type resin), a light curable resin, or a heat-curable resin can be used. The light-emitting panel may be filled with an inert gas such as nitrogen or argon, or resin such as a polyvinyl chloride (PVC) resin, an acrylic resin, a polyimide resin, an epoxy resin, a silicone resin, a polyvinyl butyral (PVB) resin, or an ethylene vinyl acetate (EVA) resin. A drying agent may be contained in the resin.

The above is the description of the material of the light-emitting panel.

At least part of this embodiment can be implemented in combination with any of the embodiments described in this specification as appropriate.

### Embodiment 3

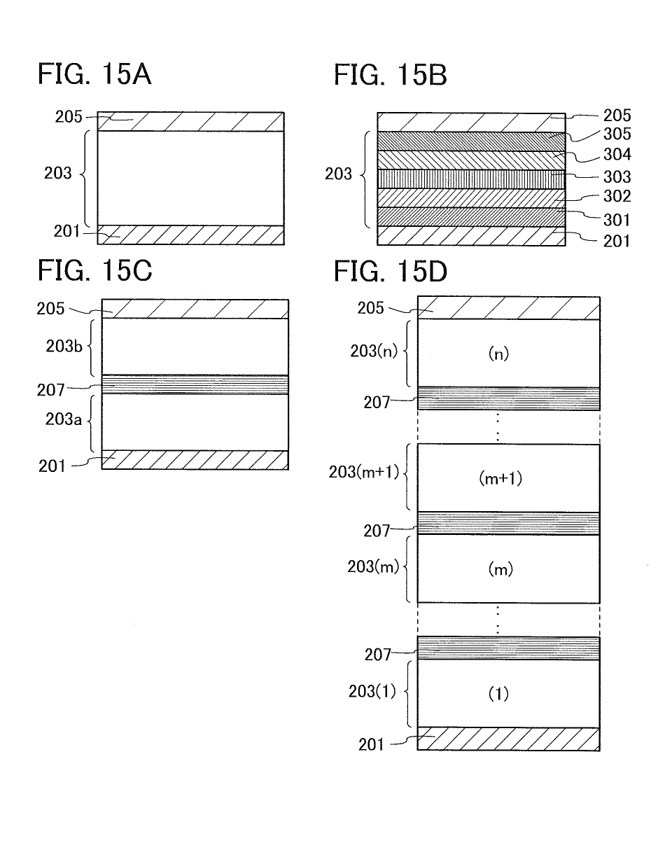

In this embodiment, light-emitting elements that can be used in the light-emitting device of one embodiment of the present invention are described with reference to FIGS. 15A to 15D.

### Structural Example of Light-Emitting Element

A light-emitting element illustrated in FIG. 15A includes an EL layer 203 between a first electrode 201 and a second electrode 205. In this embodiment, the first electrode 201 serves as the anode, and the second electrode 205 serves as the cathode.

When a voltage higher than the threshold voltage of the light-emitting element is applied between the first electrode 201 and the second electrode 205, holes are injected to the EL layer 203 from the first electrode 201 side and electrons are injected to the EL layer 203 from the second electrode 205 side. The injected electrons and holes are recombined in the EL layer 203 and a light-emitting material contained in the EL layer 203 emits light.

The EL layer 203 includes at least a light-emitting layer 303 containing a light-emitting substance.

In addition to the light-emitting layer, the EL layer 203 may further include one or more layers containing any of a substance with a high hole-injection property, a substance with a high hole-transport property, a substance with a high electron-transport property, a substance with a high electron-injection property, a substance with a bipolar property (a substance with a high electron-transport property and a high hole-transport property), and the like. For the EL layer 203, either a low molecular compound or a high molecular compound can be used, and an inorganic compound may also be used.

A light-emitting element illustrated in FIG. 15B includes the EL layer 203 between the first electrode 201 and the second electrode 205, and in the EL layer 203, a hole-injection layer 301, a hole-transport layer 302, the light-emitting layer 303, an electron-transport layer 304, and an electron-injection layer 305 are stacked in this order from the first electrode 201 side.

As in light-emitting elements illustrated in FIGS. 15C and 15D, a plurality of EL layers may be stacked between the first electrode 201 and the second electrode 205. In that case, an intermediate layer 207 is preferably provided between the stacked EL layers. The intermediate layer 207 includes at least a charge-generation region.

For example, the light-emitting element illustrated in FIG. 15C includes the intermediate layer 207 between a first EL layer 203a and a second EL layer 203b. The light-emitting element illustrated in FIG. 15D includes n EL layers (n is a natural number of 2 or more), and the intermediate layers 207 between the EL layers.

The behaviors of electrons and holes in the intermediate layer 207 provided between the EL layer 203(m) and the EL layer 203(m+1) are described below. When a voltage higher than the threshold voltage of the light-emitting element is applied between the first electrode 201 and the second electrode 205, holes and electrons are generated in the intermediate layer 207, and the holes move into the EL layer 203(m+1) provided on the second electrode 205 side and the electrons move into the EL layer 203(m) provided on the first electrode 201 side. The holes injected into the EL layer 203(m+1) are recombined with the electrons injected from the second electrode 205 side, so that a light-emitting material contained in the EL layer 203(m+1) emits light. The electrons injected into the EL layer 203(m) are recombined with the holes injected from the first electrode 201 side, so that a light-emitting material contained in the EL layer 203(m) emits light. Thus, the holes and electrons generated in the intermediate layer 207 cause light emission in the respective EL layers.

Note that the EL layers can be provided in contact with each other with no intermediate layer therebetween when these EL layers allow the same structure as the intermediate layer to be formed therebetween. For example, when the charge-generation region is formed over one surface of an EL layer, another EL layer can be provided in contact with the surface.

When the EL layers have different emission colors, a desired emission color can be obtained from the whole light-emitting element. For example, in the light-emitting element having two EL layers, when an emission color of the first EL layer and an emission color of the second EL layer are made to be complementary colors, a light-emitting element emitting white light as a whole light-emitting element can also be obtained. This can be applied to a light-emitting element including three or more EL layers.

### [Material of Light-Emitting Element]

Examples of materials that can be used for each layer are given below. Note that each layer is not limited to a single layer and may be a stack of two or more layers.

### [Anode]

The electrode serving as the anode (the first electrode 201) can be formed using one or more kinds of conductive metals, alloys, conductive compounds, and the like. In particular, it is preferable to use a material with a high work function (4.0 eV or more). Examples include indium tin oxide (ITO), indium tin oxide containing silicon or silicon oxide, indium zinc oxide, indium oxide containing tungsten oxide and zinc oxide, graphene, gold, platinum, nickel, tungsten, chromium, molybdenum, iron, cobalt, copper, palladium, and a nitride of a metal material (e.g., titanium nitride).

When the anode is in contact with the charge-generation region, any of a variety of conductive materials can be used regardless of their work functions; for example, aluminum, silver, and an alloy containing aluminum can be used.

### [Cathode]

The electrode serving as the cathode (the second electrode 205) can be formed using one or more kinds of conductive metals, alloys, conductive compounds, and the like. In particular, it is preferable to use a material with a low work function (3.8 eV or less). Examples include aluminum, silver, an element belonging to Group 1 or 2 of the periodic table (e.g., an alkali metal such as lithium and cesium, an alkaline earth metal such as calcium and strontium, and magnesium), an alloy containing any of these elements (e.g., Mg—Ag or Al—Li), a rare earth metal such as europium or ytterbium, and an alloy containing any of these rare earth metals.

Note that when the cathode is in contact with the charge-generation region, a variety of conductive materials can be used regardless of its work function. For example, ITO, indium tin oxide containing silicon or silicon oxide, or the like can be used.

The electrodes may be formed separately by a vacuum evaporation method or a sputtering method. Alternatively, when a silver paste or the like is used, a coating method or an inkjet method may be used.

### [Hole-Injection Layer 301]

The hole-injection layer 301 contains a substance with a high hole-injection property.

Examples of the substance with a high hole-injection property include metal oxides such as molybdenum oxide, vanadium oxide, ruthenium oxide, tungsten oxide, and manganese oxide; and phthalocyanine-based compounds such as phthalocyanine (abbreviation: H₂Pc) and copper(II) phthalocyanine (abbreviation: CuPc).

Other examples of the substance with a high hole-injection property include high molecular compounds such as poly(N-vinylcarbazole) (abbreviation: PVK), poly(4-vinyltriphenylamine) (abbreviation: PVTPA); and high molecular compounds to which acid is added such as poly(3,4-ethylenedioxythiophene)/poly(styrenesulfonic acid) (PEDOT/PSS).

The hole-injection layer 301 may serve as the charge-generation region. When the hole-injection layer 301 in contact with the anode serves as the charge-generation region, a variety of conductive materials can be used for the anode regardless of their work functions. Materials contained in the charge-generation region are described below.

### [Hole-Transport Layer 302]

The hole-transport layer 302 contains a substance with a high hole-transport property.

The substance with a high hole-transport property is preferably a substance with a property of transporting more holes than electrons, and is especially preferably a substance with a hole mobility of 10⁻⁶ cm²/Vs or more. A variety of compounds can be used. For example, an aromatic amine compound such as 4,4′-bis[N-(1-naphthyl)-N-phenylamino]biphenyl (abbreviation: NPB or α-NPD) or 4-phenyl-4′-(9-phenylfluoren-9-yl)triphenylamine (abbreviation: BPAFLP); a carbazole derivative such as 4,4′-di(N-carbazolyl)biphenyl (abbreviation: CBP), 9-[4-(10-phenyl-9-anthryl)phenyl]-9H-carbazole (abbreviation: CzPA), or 9-phenyl-3-[4-(10-phenyl-9-anthryl)phenyl]-9H-carbazole (abbreviation: PCzPA); an aromatic hydrocarbon compound such as 2-tert-butyl-9,10-di(2-naphthyl)anthracene (abbreviation: t-BuDNA), 9,10-di(2-naphthyl)anthracene (abbreviation: DNA), or 9,10-diphenylanthracene (abbreviation: DPAnth); a high molecular compound such as PVK or PVTPA.

### [Light-Emitting Layer 303]

For the light-emitting layer 303, a fluorescent compound that exhibits fluorescence or a phosphorescent compound that exhibits phosphorescence can be used.

Examples of the fluorescent compound that can be used for the light-emitting layer 303 include N,N-bis[4-(9H-carbazol-9-yl)phenyl]-N,N-diphenylstilbene-4,4′-diamine (abbreviation: YGA2S), N-(9,10-diphenyl-2-anthryl)-N, 9-diphenyl-9H-carbazol-3-amine (abbreviation: 2PCAPA), and rubrene.

Examples of the phosphorescent compound that can be used for the light-emitting layer 303 include organometallic complexes such as bis[2-(4′,6′-difluorophenyl)pyridinato-N,C^{2′}]iridium(III) picolinate (abbreviation: FIrpic), tris(2-phenylpyridinato-N,C^{2′})iridium(III) (abbreviation: Ir(ppy)₃), and (acetylacetonato)bis(3,5-dimethyl-2-phenylpyrazinato)iridium(III) (abbreviation: Ir(mppr-Me)₂(acac)).

The light-emitting layer 303 may have a structure in which any of the above-described light-emitting organic compounds (a light-emitting substance or a guest material) is dispersed in another substance (a host material). As the host material, a variety of kinds of materials can be used, and it is preferable to use a substance that has a lowest unoccupied molecular orbital level (LUMO level) higher than that of the guest material and has a highest occupied molecular orbital level (HOMO level) lower than that of the guest material.

With the structure in which the guest material is dispersed in the host material, crystallization of the light-emitting layer 303 can be suppressed. In addition, concentration quenching due to high concentration of the guest material can be suppressed.

As the host material, the above-described substance with a high hole-transport property (e.g., an aromatic amine compound or a carbazole derivative) or a later-described substance with a high electron-transport property (e.g., a metal complex having a quinoline skeleton or a benzoquinoline skeleton or a metal complex having an oxazole-based or thiazole-based ligand) can be used. As the host material, specifically, a metal complex such as tris(8-quinolinolato)aluminum(III) (abbreviation: Alq) or bis(2-methyl-8-quinolinolato)(4-phenylphenolato)aluminum(III) (abbreviation: BAIq); a heterocyclic compound such as 3-(4-biphenylyl)-4-phenyl-5-(4-tert-butylphenyl)-1,2,4-triazole (abbreviation: TAZ), bathophenanthroline (abbreviation: BPhen), or bathocuproine (abbreviation: BCP); a condensed aromatic compound such as CzPA, DNA, t-BuDNA, and DPAnth; or an aromatic amine compound such as NPB can be used.

Alternatively, as the host material, a plurality of kinds of materials can be used. For example, in order to suppress crystallization, a substance such as rubrene that suppresses crystallization may be further added. In addition, NPB, Alq, or the like may be further added in order to transfer energy to the guest material more efficiently.

When a plurality of light-emitting layers are provided and emission colors of the layers are made different, light emission of a desired color can be obtained from the light-emitting element as a whole. For example, in a light-emitting element having two light-emitting layers, the emission colors of first and second light-emitting layers are complementary, so that the light-emitting element can emit white light as a whole. This can be applied to a light-emitting element including three or more light-emitting layers.

### [Electron-Transport Layer 304]

The electron-transport layer 304 contains a substance with a high electron-transport property.

The substance with a high electron-transport property is preferably an organic compound having a property of transporting more electrons than holes, and is especially preferably a material with an electron mobility of 10⁻⁶ cm²/Vs or more.

As the substance with a high electron-transport property, for example, a metal complex having a quinoline skeleton or a benzoquinoline skeleton, such as Alq or Balq, can be used. Alternatively, a metal complex having an oxazole-based ligand or a thiazole-based ligand, such as bis[2-(2-hydroxyphenyl)benzoxazolato]zinc (abbreviation: Zn(BOX)₂) and bis[2-(2-hydroxyphenyl)benzothiazolato]zinc (abbreviation: Zn(BTZ)₂) can be used. Alternatively, TAZ, BPhen, BCP, or the like can be used.

### [Electron-Injection Layer 305]

The electron-injection layer 305 contains a substance with a high electron-injection property.

Examples of the substance with a high electron-injection property include alkali metals, alkaline earth metals, and compounds thereof, such as lithium, cesium, calcium, lithium fluoride, cesium fluoride, calcium fluoride, and lithium oxide. A rare earth metal compound such as erbium fluoride can also be used. Any of the above substances for the electron-transport layer 304 can also be used.

### [Charge-Generation Region]

The charge-generation region may have either a structure in which an electron acceptor (acceptor) is added to an organic compound with a high hole-transport property or a structure in which an electron donor (donor) is added to an organic compound with a high electron-transport property. Alternatively, these structures may be stacked.

Examples of the organic compound with a high hole-transport property include the above materials that can be used for the hole-transport layer, and examples of the organic compound with a high electron-transport property include the above materials that can be used for the electron-transport layer.

As examples of the electron acceptor, 7,7,8,8-tetracyano-2,3,5,6-tetrafluoroquinodimethane (abbreviation: F4-TCNQ), chloranil, and the like can be given. In addition, transition metal oxides can be given. Moreover, oxides of metals belonging to Groups 4 to 8 of the periodic table can be given. Specifically, it is preferable to use vanadium oxide, niobium oxide, tantalum oxide, chromium oxide, molybdenum oxide, tungsten oxide, manganese oxide, and rhenium oxide because of their high electron accepting properties. Among these, molybdenum oxide is especially preferable since it is stable in the air, has a low hygroscopic property, and is easy to handle.

As the electron donor, it is possible to use an alkali metal, an alkaline earth metal, a rare earth metal, a metal belonging to Group 13 of the periodic table, or an oxide or a carbonate thereof. Specifically, lithium, cesium, magnesium, calcium, ytterbium, indium, lithium oxide, cesium carbonate, or the like is preferably used. Alternatively, an organic compound such as tetrathianaphthacene may be used as the electron donor.

The above-described layers included in the EL layer 203 and the intermediate layer 207 can be formed by any of the following methods: an evaporation method (including a vacuum evaporation method), a transfer method, a printing method, an inkjet method, a coating method, and the like.

The above is the description of the material of the light-emitting element.

At least part of this embodiment can be implemented in combination with any of the embodiments described in this specification as appropriate.

### Example 1

In this example, a light-emitting panel of one embodiment of the present invention was fabricated.

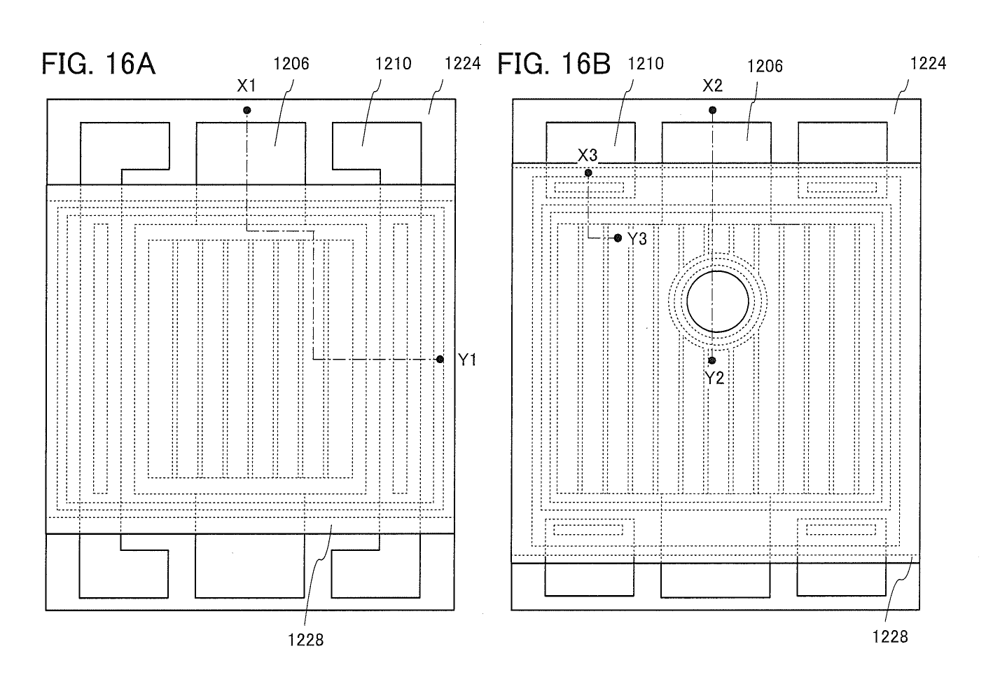

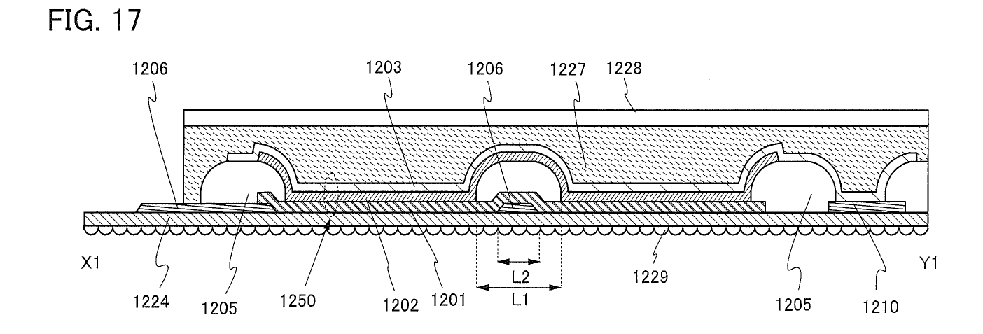

FIG. 16A is a plan view of a light-emitting panel fabricated in this example, and FIG. 17 is a cross-sectional view taken along the dashed-dotted line X1-Y1 in FIG. 16A. Note that some components of the light-emitting panel are omitted in FIG. 16A.

In the light-emitting panel of this example, as illustrated in FIG. 17, the light-emitting element 1250 is provided over the support substrate 1229 having an outcoupling structure with the insulating film 1224 therebetween. The auxiliary wiring 1206 is provided over the insulating film 1224 and is electrically connected to the first electrode 1201. The auxiliary wiring 1206 is partly exposed and functions as a terminal. The conductive layer 1210 is electrically connected to the second electrode 1203. An end portion of the first electrode 1201 and an end portion of a conductive layer 1210 are covered with a partition wall 1205. In addition, the partition wall 1205 is provided to cover the auxiliary wiring 1206 with the first electrode 1201 therebetween. The light-emitting element 1250 is sealed with the support substrate 1229, the sealing substrate 1228, and the sealant 1227.

In the light-emitting panel of this example, a diffusion film of a polyester-based resin was used as the support substrate 1229, and a substrate including a thin glass layer and a polyethylene terephthalate (PET) layer was used as the sealing substrate 1228. These substrates are flexible, and the light-emitting panel of this example is a flexible light-emitting panel. The area of a light-emitting region of the light-emitting panel of this example is 56 mm×42 mm.

The light-emitting element 1250 is an organic EL element having a bottom-emission structure; specifically, the first electrode 1201 that transmits visible light is provided over the support substrate 1229, the EL layer 1202 is provided over the first electrode 1201, and the second electrode 1203 that reflects visible light is provided over the EL layer 1202.

A method for fabricating the light-emitting panel of this example is described.

First, a base film, a separation layer (a tungsten film), and a layer to be separated were formed in this order over a glass substrate that was a formation substrate. In this example, the layer to be separated includes the insulating film 1224, the auxiliary wiring 1206, the first electrode 1201, and the partition wall 1205.

A total of seven auxiliary wirings 1206 were formed over the insulating film 1224. At this time, the auxiliary wirings 1206 with a width L2 of 322 μm were formed at a pitch of 5.3 mm. As the first electrode 1201, a film of indium tin oxide containing silicon oxide (ITSO) was formed. A total of seven partition walls 1205 covering the auxiliary wirings 1206 were formed to have a width L1 of 330 μm.

Then, a temporary support substrate and the first electrode 1201 were attached to each other with an adhesive for separation. Then, the layer to be separated was separated from the formation substrate along the separation layer. Thus, the separated layer was provided on the temporary support substrate side.

Next, the layer that was separated from the formation substrate and where the insulating film 1224 was exposed was attached to the support substrate 1229 using a UV curable adhesive. As the support substrate 1229, a diffusion film of a polyester-based resin was used as described above. Then, the temporary support substrate was separated, whereby the first electrode 1201 was exposed over the support substrate 1229.

Next, the EL layer 1202 and the second electrode 1203 were formed over the first electrode 1201. As the EL layer 1202, a first EL layer including a light-emitting layer containing a fluorescent compound emitting blue light, an intermediate layer, and a second EL layer including a light-emitting layer containing a phosphorescent compound emitting green light and a light-emitting layer containing a phosphorescent compound emitting red light were stacked in this order from the first electrode 1201 side. Silver was used for the second electrode 1203.

Then, a photo-curable resin containing zeolite that serves as the sealant 1227 was applied and cured by UV light irradiation. Next, the support substrate 1229 and the substrate including the thin glass layer and the polyethylene terephthalate (PET) layer that was the sealing substrate 1228 were attached to each other with a UV curable adhesive.

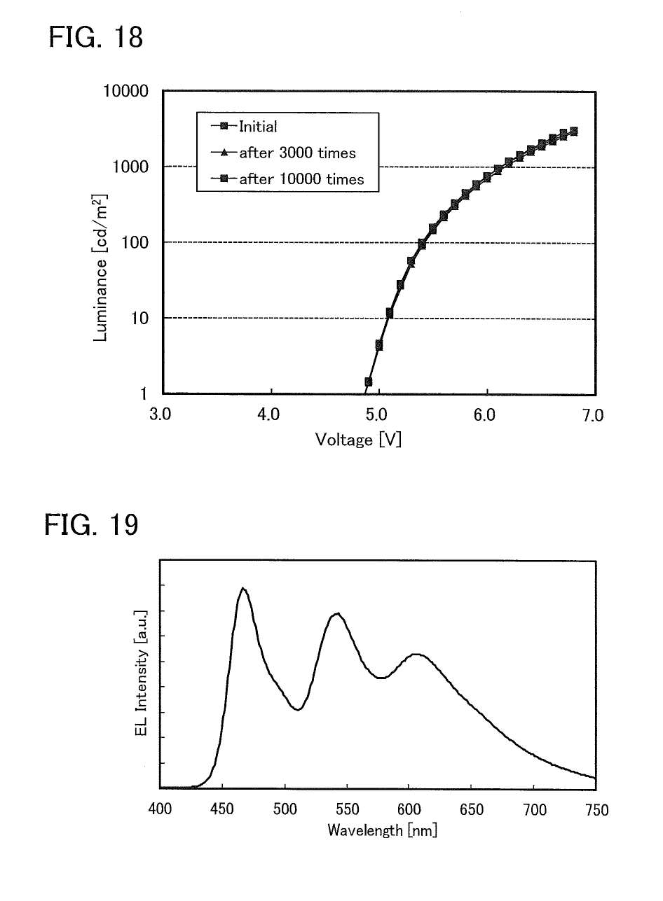

Operation characteristics of the light-emitting panel obtained in the above-described manner were measured. Voltage-luminance characteristics of the light-emitting panel are shown in FIG. 18 as indicated by “initial” in a legend. An emission spectrum of the light-emitting panel is shown in FIG. 19. It is found from FIG. 19 that the light-emitting panel of this example shows an emission spectrum including light originating from the fluorescent compound emitting blue light, light originating from the phosphorescent compound emitting green light, and light originating from the phosphorescent compound emitting red light.

After that, a light-emitting device including the light-emitting panel was subjected to a reliability test. In the reliability test, the light-emitting panel was made to emit light 3000 times or 10000 times with intervals. For each time of light emission, a current of 2 A was passed through the light-emitting panel for 50 milliseconds (ms). The current density of the light-emitting element at this time was 90 mAkm². The interval between light emissions (i.e., non-light-emitting period) was 10 seconds.

FIG. 18 shows voltage-luminance characteristics of the light-emitting panel after 3000 times of light emission and those after 10000 times of light emission.

It can be seen from FIG. 18 that the voltage-luminance characteristics of the light-emitting panel even after 10000 times of light emission does not significantly differ from those before the reliability test and that the light-emitting panel does not deteriorate. This supports that the light-emitting panel of this example has high reliability.

### Example 2

In this measurement, the amount of current that can be fed to an organic EL element emitting white light was measured. The area of a light-emitting region in the organic EL element that was used was 2 mm×2 mm. For each time of light emission, current was fed to the organic EL element for 50 milliseconds (ms).

The examination showed that a current of 60 mA was able to be fed to the organic EL element (i.e., the current density was 1500 mA/cm²). However, when a current of 68 mA was fed to the organic EL element (i.e., the current density was 1700 mA/cm²), the organic EL element was short-circuited.

The above-described results indicate that in a light-emitting device of one embodiment of the present invention that includes the organic EL element, the amount of light can be adjusted when the current density is lower than 1700 mA/cm². Thus, a larger amount of current can be fed to the organic EL element than to a light-emitting diode or the like using an inorganic material.

### Example 3

In this example, a light-emitting device of one embodiment of the present invention was fabricated.

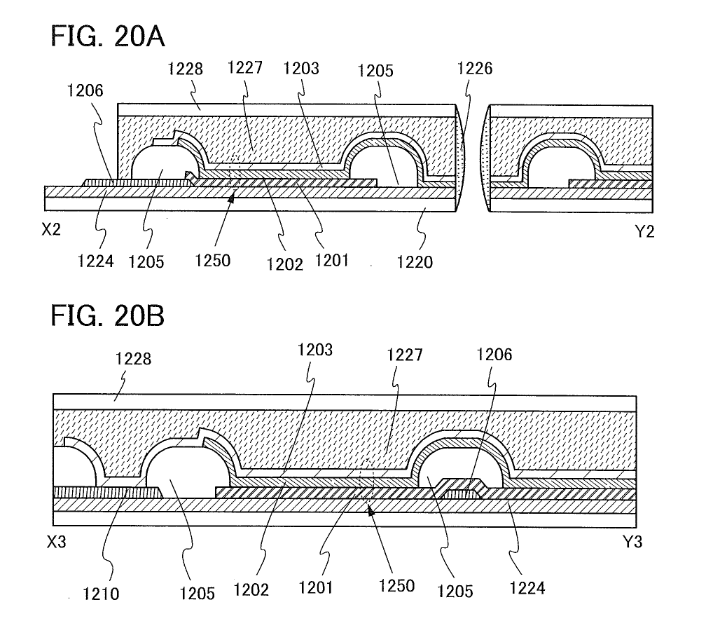

FIG. 16B is a plan view of a light-emitting panel fabricated in this example, FIG. 20A is a cross-sectional view taken along the dashed-dotted line X2-Y2 in FIG. 16B, and FIG. 20B is a cross-sectional view taken along the dashed-dotted line X3-Y3 in FIG. 16B. Note that some components of the light-emitting panel are omitted in FIG. 16B.

In the light-emitting panel of this example, the light-emitting element 1250 is formed over the support substrate 1220 with the insulating film 1224 therebetween. The auxiliary wiring 1206 is provided over the insulating film 1224 and is electrically connected to the first electrode 1201. The auxiliary wiring 1206 is partly exposed and functions as a terminal. An end portion of the first electrode 1201 and an end portion of the conductive layer 1210 are covered with the partition wall 1205. In addition, the partition wall 1205 is provided to cover the auxiliary wiring 1206 with the first electrode 1201 therebetween. The light-emitting element 1250 is sealed with the support substrate 1220, the sealing substrate 1228, and the sealant 1227.

In the light-emitting panel of this example, a diffusion film of a polyester-based resin was used as the support substrate 1220, and a substrate including a thin glass layer and a polyethylene terephthalate (PET) layer was used as the sealing substrate 1228. These substrates are flexible, and the light-emitting panel of this example is a flexible light-emitting panel. Note that it can be said that the support substrate 1220 of this example has an outcoupling structure.

A light-emitting region in the light-emitting panel of this example is obtained by excluding a circular non-light-emitting region with a diameter of 20 mm from an area of 50 mm×52.9 mm. The non-light-emitting region includes an opening of the light-emitting panel. The non-light-emitting region does not include the auxiliary wiring 1206 and the first electrode 1201 (see FIG. 20A). This structure can prevent the first electrode 1201 of the light-emitting element 1250 or the auxiliary wiring 1206 from being in contact with the second electrode 1203 and being short-circuited when an opening is formed.

The light-emitting element 1250 is an organic EL element having a bottom-emission structure; specifically, the first electrode 1201 transmitting visible light is provided over the support substrate 1220, an EL layer 1202 is provided over the first electrode 1201, and a second electrode 1203 reflecting visible light is provided over the EL layer 1202.

A method for fabricating the light-emitting panel of this example is described.

First, a base film, a separation layer (a tungsten film), and a layer to be separated were formed in this order over a glass substrate that was a formation substrate. In this example, the layer to be separated includes the insulating film 1224, the auxiliary wiring 1206, the first electrode 1201, and the partition wall 1205.

A total of 125 auxiliary wirings 1206 were formed over the insulating film 1224. At this time, the auxiliary wirings 1206 with a width L2 of 3 μm were formed at a pitch of 420 μm. As the first electrode 1201, a film of indium tin oxide containing silicon oxide (ITSO) was formed. A total of 125 partition walls 1205 covering the auxiliary wirings 1206 were formed to have a width L1 of 6 μm. The auxiliary wirings in the light-emitting panel of this example have a width as narrow as 3 μm, and thus are less likely to be recognized when the light-emitting panel emits light.

Then, a temporary support substrate and the first electrode 1201 were attached to each other with an adhesive for separation. Then, the layer to be separated was separated from the formation substrate along the separation layer. Thus, the layer to be separated is provided on the temporary support substrate side.

Next, the layer that was separated from the formation substrate and where the insulating film 1224 was exposed was attached to the support substrate 1220 with a UV curable adhesive. As the support substrate 1220, a diffusion film of a polyester-based resin was used as described above. Then, the temporary support substrate was separated, whereby the first electrode 1201 was exposed over the support substrate 1229.

Next, the EL layer 1202 and the second electrode 1203 were formed over the first electrode 1201. As the EL layer 1202, a first EL layer including a light-emitting layer containing a fluorescent compound emitting blue light, an intermediate layer, and a second EL layer including a light-emitting layer containing a phosphorescent compound emitting green light and a light-emitting layer containing a phosphorescent compound emitting orange light were stacked in this order from the first electrode 1201 side. Silver was used for the second electrode 1203.

Then, a UV curable resin containing zeolite that served as the sealant 1227 was applied and cured by UV light irradiation. Next, the support substrate 1220 and the substrate including the thin glass layer and the polyethylene terephthalate (PET) layer that was the sealing substrate 1228 were attached to each other with a UV curable adhesive.

Then, a circular opening was formed to overlap a non-light-emitting region surrounded by the light-emitting region. In this example, the opening is fainted in part of the light-emitting panel with laser light having a wavelength in the UV region (i.e., UV laser light). The opening can be formed with a punch or the like instead of laser light, in which case peeling of a film, especially the EL layer 1202 or the like, might occur because of pressure applied to the light-emitting panel. Laser light is preferably used to form the opening, in which case peeling of a film can be prevented and a highly reliable light-emitting panel can be fabricated.

Then, an end portion of the light-emitting panel that was exposed in the opening was covered with a UV curable adhesive, and the sealant 1226 was provided.

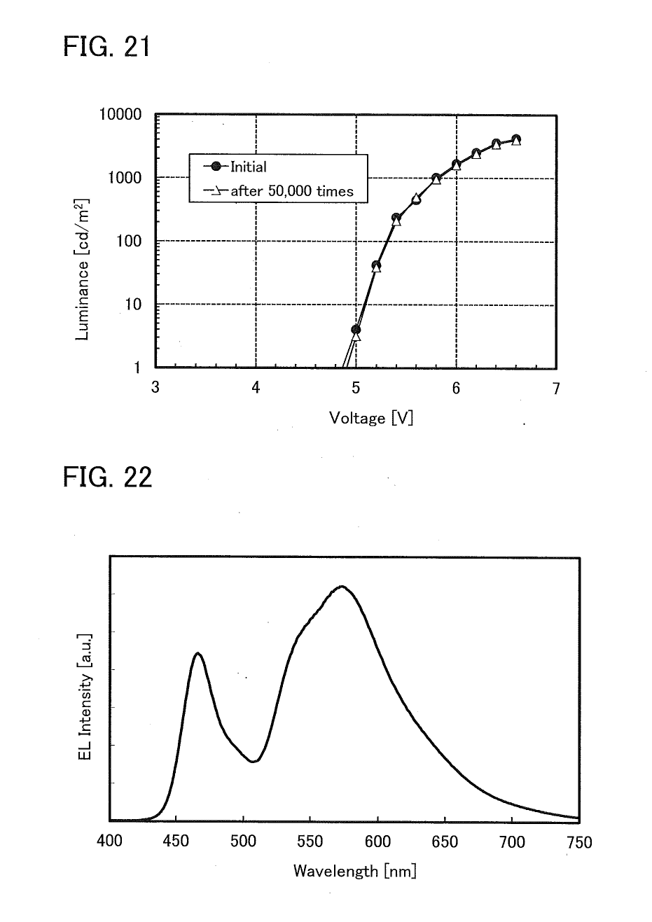

Operation characteristics of the light-emitting panel obtained in the above-described manner were measured. Voltage-luminance characteristics of the light-emitting panel are shown in FIG. 21 as indicated by “initial” in a legend. An emission spectrum of the light-emitting panel is shown in FIG. 22. It is found from FIG. 22 that the light-emitting panel of this example shows an emission spectrum including light originating from the fluorescent compound emitting blue light, light originating from the phosphorescent compound emitting green light, and light originating from the phosphorescent compound emitting orange light.

Note that the light-emitting panel emits light at a luminance of approximately 100000 cd/m² when supplied with a current of 2 A.

After that, a light-emitting device including the light-emitting panel was subjected to a reliability test. In the reliability test, the light-emitting panel was made to emit light 50000 times with intervals. For each time of light emission, a current of 2 A was fed to the light-emitting panel for 50 milliseconds (ms). The current density of the light-emitting element at this time was 87 mA/cm². The interval between light emissions (non-light-emitting period) was 0.5 seconds (s).

FIG. 21 shows voltage-luminance characteristics of the light-emitting panel after 50000 times of light emission.

It can be seen from FIG. 21 that the voltage-luminance characteristics of the light-emitting panel even after 50000 times of light emission does not significantly differ from those before the reliability test and that the light-emitting panel does not deteriorate. It is indicated that even when the light-emitting panel is made to blink for 50 milliseconds 50000 times at intervals of 0.5 seconds, heat generation due to light emission has little influence on the light-emitting panel because the actual lighting time of the light-emitting panel is only approximately 40 minutes.

At least part of this example can be implemented in combination with any of the embodiments described in this specification as appropriate.

This application is based on Japanese Patent Application serial no. 2013-188612 filed with Japan Patent Office on Sep. 11, 2013, the entire contents of which are hereby incorporated by reference.

What is claimed is:

1. An endoscope apparatus comprising:
a tubular insertion portion including an end portion,
wherein the end portion includes a light-emitting device and an optical device,
wherein the optical device is placed on an end surface of the end portion, and
wherein the light-emitting device is positioned along a side surface of the end portion.

2. The endoscope apparatus according to claim 1, wherein the light-emitting device is a surface light-emitting device.

3. The endoscope apparatus according to claim 1, wherein the optical device is an image sensor or an optical fiber.

4. The endoscope apparatus according to claim 1, wherein an outer substrate of the light-emitting device has a light-transmitting property.

5. The endoscope apparatus according to claim 1,
wherein the light-emitting device comprises a first substrate, a second substrate and a light-emitting material between the first substrate and the second substrate, and
wherein each of the first substrate and the second substrate has a light-transmitting property.

6. The endoscope apparatus according to claim 1, wherein the light-emitting device comprises an organic electroluminescence element.

7. The endoscope apparatus according to claim 1, further comprising a reflection member reflecting light.

8. The endoscope apparatus according to claim 1, wherein the light-emitting device is positioned to cover part of the end surface of the end portion.

9. An endoscope apparatus comprising:
a tubular insertion portion including an end portion,
wherein the end portion includes a light-emitting device and an optical device,
wherein the optical device is placed on an end surface of the end portion, and
wherein the light-emitting device is a surface light-emitting device.

10. The endoscope apparatus according to claim 9, wherein the optical device is an image sensor or an optical fiber.

11. The endoscope apparatus according to claim 9, wherein an outer substrate of the light-emitting device has a light-transmitting property.

12. The endoscope apparatus according to claim 9,
wherein the light-emitting device comprises a first substrate, a second substrate and a light-emitting material between the first substrate and the second substrate, and
wherein each of the first substrate and the second substrate has a light-transmitting property.

13. The endoscope apparatus according to claim 9, wherein the light-emitting device comprises an organic electroluminescence element.

14. The endoscope apparatus according to claim 9, further comprising a reflection member reflecting light.

15. The endoscope apparatus according to claim 9, wherein the light-emitting device comprises a flexible substrate.

16. An endoscope apparatus comprising:
a tubular insertion portion including an end portion,
wherein the end portion includes a light-emitting device and an optical device,
wherein the optical device is placed on an end surface of the end portion,
wherein the light-emitting device is a surface light-emitting device,
wherein an image obtained through the optical device has a first mode to observe a moving image and a second mode to take a still image,
wherein in the first mode, light whose emission luminance is greater than or equal to 100 cd/cm² and less than 10000 cd/cm² is emitted continuously or intermittently, and
wherein in the second mode, light whose emission luminance is greater than or equal to 10000 cd/cm² and less than or equal to 500000 cd/cm² is emitted by being synchronized with shutter speed.

17. The endoscope apparatus according to claim 16, wherein the optical device is an image sensor or an optical fiber.

18. The endoscope apparatus according to claim 16, wherein an outer substrate of the light-emitting device has a light-transmitting property.

19. The endoscope apparatus according to claim 16,
wherein the light-emitting device comprises a first substrate, a second substrate and a light-emitting material between the first substrate and the second substrate, and
wherein each of the first substrate and the second substrate has a light-transmitting property.

20. The endoscope apparatus according to claim 16, wherein the light-emitting device comprises an organic electroluminescence element.

21. The endoscope apparatus according to claim 16, further comprising a reflection member reflecting light.

22. The endoscope apparatus according to claim 16, wherein the light-emitting device comprises a flexible substrate.
